SEQUENCE LISTING (72 sequences)

SEQ ID NO: 1 - DNA, 3810 bp, Artificial sequence
  Synthetic polynucleotide
  misc_feature: MegaTAL-CCR5-furin-T2A-tEFGR DNA sequence
atgggatcctgcaggtatccatatgatgtcccagattatgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttcagacgaaaaagaggaagcggagagggcagaggcagtctgctgacatgcggtgacgtggaagagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 2 - DNA, 2661 bp, Artificial sequence
  Synthetic polynucleotide
  misc_feature: nuclease coding sequence of SEQ ID NO1
atgggatcctgcaggtatccatatgatgtcccagattatgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttc

SEQ ID NO: 3 - DNA, 75 bp, Artificial sequence
  Synthetic polynucleotide
  misc_feature: furin and ribosomal skip sequence of SEQ ID NO 1
agacgaaaaagaggaagcggagagggcagaggcagtctgctgacatgcggtgacgtggaagagaatcccggccct

SEQ ID NO: 4 - DNA, 1074 bp, Artificial sequence
  Synthetic polynucleotide
  misc_feature: tEGFR sequence of SEQ ID NO1
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 5 - protein, 1269 aa, Artificial sequence
  Synthetic polypeptide
  misc_feature: MegaTAL-CCR5-furin-T2A-tEFGR amino acid sequence
MGSCRYPYDVPDYAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSRRESINPWILTGFTDAEGSFMLRIRNTNNRSVGYYTSLVFEITLHNKDKSILENIQSTWKVGTINNRGDGTARLSVTRFEDLKVIIDHFEKYPLITQKLGDYKLFKQAFSVMENKEHLKENGIKELVRIKAKMNWGLNDELKKAFPENISKERPLINKNIPNLKWLAGFTSGEGTFYVHLAKSEASGKVYVRLRFIIGQHIRDKNLMNSLITYLGCGTIQEKNRSKGSMLHFIVTKFSDINDKIIPVFQENTLIGVKLEDFEDWCKVAKLIEEKKHLTESGLDEIKKIKLNMNKGRVFRRKRGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 6 - protein, 887 aa, Artificial sequence
  Synthetic polypeptide
  misc_feature: nuclease or base editor coding sequence of SEQ ID NO5
MGSCRYPYDVPDYAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSRRESINPWILTGFTDAEGSFMLRIRNTNNRSVGYYTSLVFEITLHNKDKSILENIQSTWKVGTINNRGDGTARLSVTRFEDLKVIIDHFEKYPLITQKLGDYKLFKQAFSVMENKEHLKENGIKELVRIKAKMNWGLNDELKKAFPENISKERPLINKNIPNLKWLAGFTSGEGTFYVHLAKSEASGKVYVRLRFIIGQHIRDKNLMNSLITYLGCGTIQEKNRSKGSMLHFIVTKFSDINDKIIPVFQENTLIGVKLEDFEDWCKVAKLIEEKKHLTESGLDEIKKIKLNMNKGRVF

SEQ ID NO: 7 - protein, 25 aa, Artificial sequence
  Synthetic polypeptide
  misc_feature: furin and ribosomal skip sequence of SEQ ID NO 5
RRKRGSGEGRGSLLTCGDVEENPGP

SEQ ID NO: 8 - protein, 357 aa, Artificial sequence
  Synthetic polypeptide
  misc_feature: tEGFR sequence of SEQ ID NO5
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 9 - DNA, 3861 bp, Artificial sequence
  Synthetic polynucleotide
  misc_feature: MegaTAL-CCR5-GAfurin-T2A-tEFGR DNA sequence
atgggatcctgcaggtatccatatgatgtcccagattatgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttcggtggggccggggcaggcgctggtcggtcccgcgcgaaaagaagcgttggcggtgcaggcgcaggtgcgggggagggcagaggcagtctgctgacatgcggtgacgtggaagagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 10 - DNA, 2661 bp, Artificial sequence; identical to SEQ ID NO: 2
  Synthetic polynucleotide
  misc_feature: nuclease or base editor coding sequence of SEQ ID NO9

SEQ ID NO: 11 - DNA, 126 bp, Artificial sequence
  Synthetic polynucleotide
  misc_feature: furin and ribosomal skip sequence of SEQ ID NO 9
ggtggggccggggcaggcgctggtcggtcccgcgcgaaaagaagcgttggcggtgcaggcgcaggtgcgggggagggcagaggcagtctgctgacatgcggtgacgtggaagagaatcccggccct

SEQ ID NO: 12 - DNA, 1074 bp, Artificial sequence; identical to SEQ ID NO: 4
  Synthetic polynucleotide
  misc_feature: tEGFR sequence of SEQ ID NO9

SEQ ID NO: 13 - protein, 1286 aa, Artificial sequence
  Synthetic polypeptide
  misc_feature: MegaTAL-CCR5-GAfurin-T2A-tEFGR amino acid sequence
MGSCRYPYDVPDYAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSRRESINPWILTGFTDAEGSFMLRIRNTNNRSVGYYTSLVFEITLHNKDKSILENIQSTWKVGTINNRGDGTARLSVTRFEDLKVIIDHFEKYPLITQKLGDYKLFKQAFSVMENKEHLKENGIKELVRIKAKMNWGLNDELKKAFPENISKERPLINKNIPNLKWLAGFTSGEGTFYVHLAKSEASGKVYVRLRFIIGQHIRDKNLMNSLITYLGCGTIQEKNRSKGSMLHFIVTKFSDINDKIIPVFQENTLIGVKLEDFEDWCKVAKLIEEKKHLTESGLDEIKKIKLNMNKGRVFGGAGAGAGRSRAKRSVGGAGAGAGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 14 - protein, 887 aa, Artificial sequence; identical to SEQ ID NO: 6
  Synthetic polypeptide
  misc_feature: nuclease or base editor coding sequence of SEQ ID NO13

SEQ ID NO: 15 - protein, 42 aa, Artificial sequence
  Synthetic polypeptide
  misc_feature: furin and ribosomal skip sequence of SEQ ID NO 13
GGAGAGAGRSRAKRSVGGAGAGAGEGRGSLLTCGDVEENPGP

SEQ ID NO: 16 - protein, 357 aa, Artificial sequence; identical to SEQ ID NO: 8
  Synthetic polypeptide
  misc_feature: tEGFR sequence of SEQ ID NO13

SEQ ID NO: 17 - DNA, 3813 bp, Artificial sequence
  Synthetic polynucleotide
  misc_feature: MegaTAL-CCR5-furin-P2A-tEFGR DNA sequence
atgggatcctgcaggtatccatatgatgtcccagattatgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttccgaaaaagaagaggaagcggagctactaacttcagcctgctgaagcaggctggcgacgtggaggagaaccctggacctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 18 - DNA, 2661 bp, Artificial sequence; identical to SEQ ID NO: 2
  Synthetic polynucleotide
  misc_feature: nuclease or base editor coding sequence of SEQ ID NO17

SEQ ID NO: 19 - DNA, 78 bp, Artificial sequence
  Synthetic polynucleotide
  misc_feature: furin and ribosomal skip sequence of SEQ ID NO 17
cgaaaaagaagaggaagcggagctactaacttcagcctgctgaagcaggctggcgacgtggaggagaaccctggacct

SEQ ID NO: 20 - DNA, 1074 bp, Artificial sequence; identical to SEQ ID NO: 4
  Synthetic polynucleotide
  misc_feature: tEGFR sequence of SEQ ID NO17

SEQ ID NO: 21 - protein, 1270 aa, Artificial sequence
  Synthetic polypeptide
  misc_feature: MegaTAL-CCR5-furin-P2A-tEFGR amino acid sequence
MGSCRYPYDVPDYAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSRRESINPWILTGFTDAEGSFMLRIRNTNNRSVGYYTSLVFEITLHNKDKSILENIQSTWKVGTINNRGDGTARLSVTRFEDLKVIIDHFEKYPLITQKLGDYKLFKQAFSVMENKEHLKENGIKELVRIKAKMNWGLNDELKKAFPENISKERPLINKNIPNLKWLAGFTSGEGTFYVHLAKSEASGKVYVRLRFIIGQHIRDKNLMNSLITYLGCGTIQEKNRSKGSMLHFIVTKFSDINDKIIPVFQENTLIGVKLEDFEDWCKVAKLIEEKKHLTESGLDEIKKIKLNMNKGRVFRKRRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 22 - protein, 887 aa, Artificial sequence; identical to SEQ ID NO: 6
  Synthetic polypeptide
  misc_feature: nuclease or base editor coding sequence of SEQ ID NO21

SEQ ID NO: 23 - protein, 26 aa, Artificial sequence
  Synthetic polypeptide
  misc_feature: furin and ribosomal skip sequence of SEQ ID NO 21
RKRRGSGATNFSLLKQAGDVEENPGP

SEQ ID NO: 24 - protein, 357 aa, Artificial sequence; identical to SEQ ID NO: 8
  Synthetic polypeptide
  misc_feature: tEGFR sequence of SEQ ID NO21

SEQ ID NO: 25 - DNA, 3456 bp, Artificial sequence
  Synthetic polynucleotide
  misc_feature: MegaTAL-CCR5-furin-T2A-eGFP DNA sequence
atgggatcctgcaggtatccatatgatgtcccagattatgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctcgcgcagagagtccatcaacccatggattctgactggtttcactgatgccgaaggatcattcatgctaagaatccgtaacacgaacaaccggtcagtagggtactacacttcactggtattcgaaatcactctgcacaacaaggacaaatcgattcttgagaatatccagtcgacttggaaggtcggcacaatcaacaaccgaggcgacggcaccgccagactgagcgtcactcgtttcgaagatttgaaagtgattatcgaccacttcgagaaatatccgctgattacccagaaattgggcgattacaagttgtttaaacaggcattcagcgtcatggagaacaaagaacatcttaaggagaatgggattaaggagctcgtacgaatcaaagctaagatgaattggggtctcaatgacgaattgaaaaaagcatttccagagaacatcagcaaagagcgcccccttatcaataagaacattccgaatctcaaatggctggctggattcacatctggtgaaggcacattctacgtgcacctagcaaagtctgaagctagcggcaaggtatacgtgcgactgaggttcataatcggccagcacatcagagacaagaacctgatgaattcattgataacatacctaggctgtggtacgatccaggagaagaacaggtctaagggcagtatgctccacttcatagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgtgtcttcagacgaaaaagaggaagcggagagggcagaggcagtctgctgacatgcggtgacgtggaagagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 26 - DNA, 2661 bp, Artificial sequence; identical to SEQ ID NO: 2
  Synthetic polynucleotide
  misc_feature: nuclease or base editor coding sequence of SEQ ID NO25

SEQ ID NO: 27 - DNA, 75 bp, Artificial sequence; identical to SEQ ID NO: 3
  Synthetic polynucleotide
  misc_feature: furin and ribosomal skip sequence of SEQ ID NO 25

SEQ ID NO: 28 - DNA, 720 bp, Artificial sequence
  Synthetic polynucleotide
  misc_feature: eGFP sequence of SEQ ID NO25
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 29 - protein, 1151 aa, Artificial sequence
  Synthetic polypeptide
  misc_feature: MegaTAL-CCR5-furin-T2A-eGFP amino acid sequence
MGSCRYPYDVPDYAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSRRESINPWILTGFTDAEGSFMLRIRNTNNRSVGYYTSLVFEITLHNKDKSILENIQSTWKVGTINNRGDGTARLSVTRFEDLKVIIDHFEKYPLITQKLGDYKLFKQAFSVMENKEHLKENGIKELVRIKAKMNWGLNDELKKAFPENISKERPLINKNIPNLKWLAGFTSGEGTFYVHLAKSEASGKVYVRLRFIIGQHIRDKNLMNSLITYLGCGTIQEKNRSKGSMLHFIVTKFSDINDKIIPVFQENTLIGVKLEDFEDWCKVAKLIEEKKHLTESGLDEIKKIKLNMNKGRVFRRKRGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - protein, 887 aa, Artificial sequence; identical to SEQ ID NO: 6
  Synthetic polypeptide
  misc_feature: nuclease or base editor coding sequence of SEQ ID NO29

SEQ ID NO: 31 - protein, 25 aa, Artificial sequence
  Synthetic polypeptide
  misc_feature: furin and ribosomal skip sequence of SEQ ID NO 29
RRKRGSGEGRGSLLTCGDVEENPGP

SEQ ID NO: 32 - protein, 239 aa, Artificial sequence
  Synthetic polypeptide
  misc_feature: eGFP sequence of SEQ ID NO29
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 33 - DNA, 5349 bp, Artificial sequence
  Synthetic polynucleotide
  misc_feature: Cas9 -furin-T2A-tEGFR DNA sequence
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagagacgaaaaagaggaagcggagagggcagaggcagtctgctgacatgcggtgacgtggaagagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 34 - DNA, 4200 bp, Artificial sequence
  Synthetic polynucleotide
  misc_feature: nuclease or base editor coding sequence of SEQ ID NO33
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 35 - DNA, 75 bp, Artificial sequence; identical to SEQ ID NO: 3
  Synthetic polynucleotide
  misc_feature: furin and ribosomal skip sequence of SEQ ID NO 33

SEQ ID NO: 36 - DNA, 1074 bp, Artificial sequence; identical to SEQ ID NO: 4
  Synthetic polynucleotide
  misc_feature: tEGFR sequence of SEQ ID NO33

SEQ ID NO: 37 - protein, 1782 aa, Artificial sequence
  Synthetic polypeptide
  misc_feature: Cas9 -furin-T2A-tEGFR amino acid sequence
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKRRKRGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 38 - protein, 1400 aa, Artificial sequence
  Synthetic polypeptide
  misc_feature: nuclease or base editor coding sequence of SEQ ID NO37
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 39 - protein, 25 aa, Artificial sequence
  Synthetic polypeptide
  misc_feature: furin and ribosomal skip sequence of SEQ ID NO 37
RRKRGSGEGRGSLLTCGDVEENPGP

SEQ ID NO: 40 - protein, 357 aa, Artificial sequence; identical to SEQ ID NO: 8
  Synthetic polypeptide
  misc_feature: tEGFR sequence of SEQ ID NO37

SEQ ID NO: 41 - DNA, 4995 bp, Artificial sequence
  Synthetic polynucleotide
  misc_feature: Cas9-furin-T2A-eGFP DNA sequence
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagagacgaaaaagaggaagcggagagggcagaggcagtctgctgacatgcggtgacgtggaagagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 42 - DNA, 4200 bp, Artificial sequence; identical to SEQ ID NO: 34
  Synthetic polynucleotide
  misc_feature: nuclease or base editor coding sequence of SEQ ID NO41

SEQ ID NO: 43 - DNA, 75 bp, Artificial sequence; identical to SEQ ID NO: 3
  Synthetic polynucleotide
  misc_feature: furin and ribosomal skip sequence of SEQ ID NO 41

SEQ ID NO: 44 - DNA, 720 bp, Artificial sequence; identical to SEQ ID NO: 28
  Synthetic polynucleotide
  misc_feature: eGFP sequence of SEQ ID NO41

SEQ ID NO: 45 - protein, 1664 aa, Artificial sequence
  Synthetic polypeptide
  misc_feature: Cas9-furin-T2A-eGFP amino acid sequence
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKRRKRGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 1400 aa, Artificial sequence; identical to SEQ ID NO: 38
  Synthetic polypeptide
  misc_feature: nuclease or base editor coding sequence of SEQ ID NO45

SEQ ID NO: 47 - protein, 25 aa, Artificial sequence
  Synthetic polypeptide
  misc_feature: furin and ribosomal skip sequence of SEQ ID NO 45
RRKRGSGEGRGSLLTCGDVEENPGP

SEQ ID NO: 48 - protein, 239 aa, Artificial sequence; identical to SEQ ID NO: 32
  Synthetic polypeptide
  misc_feature: eGFP sequence of SEQ ID NO45

SEQ ID NO: 49 - DNA, 6708 bp, Artificial sequence
  Synthetic polynucleotide
  misc_feature: AncBE3max-dCas9-furin-T2A-tEGFR DNA sequence
atgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctaaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtcagacgaaaaagaggaagcggagagggcagaggcagtctgctgacatgcggtgacgtggaagagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 50 - DNA, 5559 bp, Artificial sequence
  Synthetic polynucleotide
  misc_feature: nuclease or base editor coding sequence of SEQ ID NO49
atgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctaaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 51 - DNA, 75 bp, Artificial sequence; identical to SEQ ID NO: 3
  Synthetic polynucleotide
  misc_feature: furin and ribosomal skip sequence of SEQ ID NO 49

SEQ ID NO: 52 - DNA, 1074 bp, Artificial sequence; identical to SEQ ID NO: 4
  Synthetic polynucleotide
  misc_feature: tEGFR sequence of SEQ ID NO49

SEQ ID NO: 53 - protein, 2235 aa, Artificial sequence
  Synthetic polypeptide
  misc_feature: AncBE3-dCas9-furin-T2A-tEGFR amino acid sequence
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVRRKRGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 54 - protein, 1853 aa, Artificial sequence
  Synthetic polypeptide
  misc_feature: nuclease or base editor coding sequence of SEQ ID NO53
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 55 - protein, 25 aa, Artificial sequence
  Synthetic polypeptide
  misc_feature: furin and ribosomal skip sequence of SEQ ID NO 53
RRKRGSGEGRGSLLTCGDVEENPGP

SEQ ID NO: 56 - protein, 357 aa, Artificial sequence; identical to SEQ ID NO: 8
  Synthetic polypeptide
  misc_feature: tEGFR sequence of SEQ ID NO53

SEQ ID NO: 57 - DNA, 6558 bp, Artificial sequence
  Synthetic polynucleotide
  misc_feature: ABE7.10max-dCas9-furin-T2A-tEGFR DNA sequence
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcagacgaaaaagaggaagcggagagggcagaggcagtctgctgacatgcggtgacgtggaagagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtag

SEQ ID NO: 58 - DNA, 5409 bp, Artificial sequence
  Synthetic polynucleotide
  misc_feature: nuclease or base editor coding sequence of SEQ ID NO57
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 59 - DNA, 75 bp, Artificial sequence; identical to SEQ ID NO: 3
  Synthetic polynucleotide
  misc_feature: furin and ribosomal skip sequence of SEQ ID NO 57

SEQ ID NO: 60 - DNA, 1074 bp, Artificial sequence; identical to SEQ ID NO: 4
  Synthetic polynucleotide
  misc_feature: tEGFR sequence of SEQ ID NO57

SEQ ID NO: 61 - protein, 2185 aa, Artificial sequence
  Synthetic polypeptide
  misc_feature: ABE7.10max-dCas9-furin-T2A-tEGFR DNA sequence
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVRRKRGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 62 - protein, 1803 aa, Artificial sequence
  Synthetic polypeptide
  misc_feature: nuclease or base editor coding sequence of SEQ ID NO61
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 63 - protein, 25 aa, Artificial sequence
  Synthetic polypeptide
  misc_feature: furin and ribosomal skip sequence of SEQ ID NO61
RRKRGSGEGRGSLLTCGDVEENPGP

SEQ ID NO: 64 - protein, 357 aa, Artificial sequence; identical to SEQ ID NO: 8
  Synthetic polypeptide
  misc_feature: tEGFR sequence of SEQ ID NO61

SEQ ID NO: 65 - protein, 18 aa, Artificial sequence
  Synthetic polypeptide
  misc_feature: T2A
EGRGSLLTCGDVEENPGP

SEQ ID NO: 66 - protein, 19 aa, Artificial sequence
  Synthetic polypeptide
  misc_feature: P2A
ATNFSLLKQAGDVEENPGP

SEQ ID NO: 67 - protein, 20 aa, Artificial sequence
  Synthetic polypeptide
  misc_feature: E2A
QCTNYALLKLAGDVESNPGP

SEQ ID NO: 68 - protein, 22 aa, Artificial sequence
  Synthetic polypeptide
  misc_feature: F2A
VKQTLNFDLLKLAGDVESNPGP

SEQ ID NO: 69 - protein, 25 aa, Artificial sequence
  Synthetic polypeptide
  misc_feature: Furin-T2A
RRKRGSGEGRGSLLTCGDVEENPGP

SEQ ID NO: 70 - protein, 42 aa, Artificial sequence; identical to SEQ ID NO: 15
  Synthetic polypeptide
  misc_feature: GA-Furin-T2A

SEQ ID NO: 71 - protein, 26 aa, Artificial sequence
  Synthetic polypeptide
  misc_feature: Furin-P2A
RKRRGSGATNFSLLKQAGDVEENPGP

SEQ ID NO: 72 - protein, 24 aa, Artificial Sequence
  Synthetic polypeptide
GGAGAGAGRSRAKRSVGGAGAGAG

(19) United States
(12) Patent Application Publication
(10) Pub. No.: US 2023/0167431 A1
(43) Pub. Date: Jun. 1, 2023

(54) TAGGED GENE EDITING TECHNOLOGY FOR CLINICAL CELL SORTING AND ENRICHMENT

(71) Applicant: CITY OF HOPE, Duarte, CA (US)

(72) Inventors: Angelo CARDOSO, Duarte, CA (US); Tristan SCOTT, Duarte, CA (US); Diana BROWNING, Duarte, CA (US); Kevin MORRIS, Duarte, CA (US); John ZAIA, Duarte, CA (US)

(21) Appl. No.: 17/997,351

(22) PCT Filed: Apr. 28, 2021
(86) PCT No.: PCT/US2021/029714

§ 371 Date: Oct. 27, 2022

Related U.S. Application Data
(60) Provisional application No. 63/016,841, filed on Apr. 28, 2020.

(51) Int. Cl. C12N 15/10; A61K 35/17; A61P 31/18; C07K 14/71; C12N 9/22

(52) U.S. Cl. CPC C12N 15/102; A61K 35/17; A61P 31/18; C07K 14/71; C12N 9/22; A61K 2039/5156

(57) ABSTRACT

Provided herein, inter alia, are constructs and methods for making genetically modified cells that express truncated EGFR (tEGFR). The constructs can be used for identifying, selecting and determining efficacy of the genetically modified cells. Further provided are methods of using the genetically modified cells for treating diseases.

### CROSS-REFERENCE TO RELATED APPLICATIONS

This application claims priority to U.S. Provisional Application No. 63/016,841, filed Apr. 28, 2021, which is hereby incorporated by reference in its entirety and for all purposes.

### SEQUENCE LISTING REFERENCE TO A SEQUENCE LISTING, A TABLE OR A COMPUTER PROGRAM LISTING APPENDIX SUBMITTED AS AN ASCII TEXT FILE

The Sequence Listing written in file 048440-697001WO_SEQUENCE_LISTING_ST25.TXT, created on Apr. 28, 2021, 339,374 bytes bytes, machine format IBM-PC, MS Windows operating system, is hereby incorporated by reference.

### BACKGROUND

A cure for HIV remains an unmet medical need. HIV/AIDS affects approximately 1 million people in the United States, and lifelong antiretroviral drug therapy (ART) suppresses HIV to undetectable levels, but does not eradicate the HIV infection. ART can have serious side effects in 15-25% of patients, and the cost over a lifetime is estimated to be approximately $600,000. The Centers for Disease Control (CDC) estimates that only 55% of HIV patients achieved optimal adherence to ART in the United States.

Anti-HIV chimeric antigen receptor (CAR) T cell therapy is promising, but requires the presence of a target antigen. During ART, HIV-1 antigenemia is minimal and CAR T cells will not persist. It is challenging to determine how to safely administer CAR T cell therapy while containing the HIV infection.

CAR T cells and other genetically modified therapeutic cells are difficult to manufacture in large numbers. In addition, selection of cells containing the genetic modification is desirable for therapeutic applications.

Provided herein are, inter alia, solutions to these and other problems in the art.

### BRIEF SUMMARY

Described herein, inter alia, are compositions (e.g. nucleic acid construct) and methods for selecting genetically modified cells. The genetically modified cells described herein express truncated epidermal growth factor receptor (tEGFR), thereby allowing for identification and enrichment of said cells by detection of tEGFR. In embodiments, the selected cells are useful for treating and/or preventing diseases in a subject.

In an aspect is provided a nucleic acid construct including a polynucleotide encoding a gene editing agent, a polynucleotide encoding a linker peptide, and a polynucleotide encoding a truncated epidermal growth factor receptor (tEGFR). In embodiments, the polynucleotide encoding the linker peptide is located between the polynucleotide encoding the tEGFR and the polynucleotide encoding the gene editing agent.

In another aspect is provided an expression vector including the nucleic acid construct provided herein including embodiments thereof.

In an aspect is provided a cell including a nucleic acid construct provided herein including embodiments thereof. In another aspect is provided a cell including an expression vector provided herein including embodiments thereof.

In an aspect is provided a method of selecting for genetically modified cells from a population of cells, the method including: (a) contacting the population of cells with a nucleic acid construct including a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent; (b) growing the cells under conditions such that: (i) the gene editing agent and the tEGFR are expressed in a subset of cells, and (ii) the gene editing agent edits one or more genes in the subset of the cells, thereby forming the genetically modified cells in the population of cells; and (c) selecting tEGFR-expressing cells, thereby selecting the genetically modified cells from the population of cells.

In another aspect a method of making a genetically modified cell is provided, the method including: (a) contacting a population of cells with a nucleic acid construct including a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent, (b) growing the cells under conditions such that the gene editing agent and the tEGFR are expressed, thereby forming a tEGFR-expressing genetically modified cell; and (c) selecting the tEGFR-expressing genetically modified cell, thereby selecting the genetically modified cell from the population of cells.

In an aspect is provided a method for genetically modifying cells in a cell population, the method including: (a) contacting the cell population with a genome editing agent and a polynucleotide encoding a truncated epidermal growth factor receptor (tEGFR), thereby forming genetically modified cells within the cell population; (b) growing the cells under conditions that the tEGFR is expressed in the genetically modified cells; and (c) sorting the genetically modified cells from the cell population based on expression of tEGFR.

In another aspect is provided a method of release testing a population of cells including genetically modified cells, wherein the genetically modified cells express a truncated epidermal growth factor receptor (tEGFR) and a gene editing agent, the method including detecting an amount of tEGFR-expressing cells in the population of cells, wherein the population of cells is ready for release if at least about 2.5% of the cells in the cell population are tEGFR-expressing cells.

In another aspect is provided a method for release testing of a cell population, wherein at least a subset of the cells in the cell population have been genetically modified, and wherein the genetically modified cells express a truncated epidermal growth factor receptor (tEGFR), the method including determining the amount of cells in the cell population expressing the tEGFR and determining that the composition is ready for release.

In an aspect is provided a method of identifying genetically modified cells in a population of cells, wherein the genetically modified cells express a truncated epidermal growth factor receptor (tEGFR) and a gene editing agent, the method including detecting tEGFR-expressing cells, thereby identifying the genetically modified cells in the population of cells.

In an aspect is provided a method for identifying genetically modified cells from a cell population, wherein at least a subset of the cells in the cell population have been genetically modified, and wherein the genetically modified cells express a truncated epidermal growth factor receptor (tEGFR), the method including determining the amount of cells in the composition expressing the tEGFR.

In another aspect is provided a method of treating a disease in a subject in need thereof, the method including administering to the subject a therapeutically effect amount of a genetically modified cell provided herein including embodiments thereof.

### BRIEF DESCRIPTION OF THE DRAWINGS

FIG. 1 is a schematic of a method of making cells expressing MegaTAL with a tEGFR. The MegaTAL-tEFGR cassette includes a CMV promoter for co-expression of MegaTAL and truncated EGFR (tEGFR) separated by a 2A cleavage peptide (2A). The two proteins separate, with the MegaTAL moving to the nucleus to bind, cleave and mutate the target site while the tEGFR is trafficked to the cell surface. tEGFR can be used for sorting to enrich for CCR5-deleted cells, or quantify tEGFR as an indicator of the levels of gene editing.

FIG. 2 shows a schematic of three different constructs of MegaTAL targeting CCR5 (MegaTAL-CCR5) linked to a truncated EFGR (tEGFR) marker. Schematic and sequences of the resulting peptide sequences are shown; arrows indicate the predicted furin cleavage and ribosomal skip sites. The sequences include SEQ ID NO: 31 (top), SEQ ID NO: 70 (middle), and SEQ ID NO: 71 (bottom). The size of the open reading frame (ORF) for each construct is indicated.

FIG. 3 shows cell-surface tEGFR expression using the constructs of FIG. 2 evaluated in HEK293T cells. The graphs show (top to bottom): control (no tEGFR); furin T2A linker; furin P2A linker; GATA-furin T2A linker.

FIG. 4 shows a schematic of example nuclease and editing systems that may be fused to the tEGFR marker, including megaTAL-CCR5, a nuclease Cas9, or a nickase (nCas9-D10A) fused to a deaminase (DA), e.g., adenosine deaminase or cytosine deaminase. The size of the ORFs are indicated.

FIGS. 5A-5B show the effects of CCR5MT-tEGFR on CEM.NKR CCR5+ cells. A stable CCR5 expressing cell line (CEM.NKR CCR5+) was electroporated with the CCR5MT-tEGFR and 24 hours later stained and flow sorting selected for tEGFR positive cells. Cells were cultured for an additional week prior to T7E1 endonuclease. FIG. 5A shows InDel (insertion or deletion of bases) detection by a T7E1 assay. FIG. 5B shows CCR5 expression as determined by FACS.

FIG. 6 shows results of HIV challenge assay of T cells treated with CCR5MT-EGFR.

FIGS. 7A-7B show T7E1 assay for MegaTAL endonuclease activity in T cells treated with CCR5MT-tEGFR. DNA was isolated from cells used for the HIV challenge assay and the region of CCR5 DNA sequence targeted for disruption. FIG. 7A shows percent of cleaved DNA; FIG. 7B shows percent of estimated InDels

FIG. 8 shows that the tEGFR tag can be used for other gene editing systems. The tEGFR was fused to the Cas9 with T2A ribosomal skip sequence as shown in FIG. 4, and following electroporation with Cas9-tEGFR the expression of tEGFR was determined by FACS in K562 cells.

FIGS. 9A-9B show the effects of Cas9-tEGFR in K562 cells. The cells were electroporated with either 1 or 2 ug of the Cas-tEGFR in addition with a targeting gRNA. FIG. 9A shows detection of tEGFR expression by FACS and FIG. 9B shows percent nuclease efficiency for cells treated with 1 and 2 ug of the construct as determined by TIDE analysis.

FIGS. 10A-10B show the effects of Cas9-tEGFR in activated T cells. The activated T cells were electroporated with either 1 or 2 ug of the Cas-tEGFR in addition to a targeting gRNA. FIG. 10A shows tEGFR expression on the surface of the T cells as detected by FACS and FIG. 10B shows percent nuclease efficiency for cells treated with 1 and 2 ug of the construct as determined by TIDE analysis.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The terms “nucleic acid molecule” and “polynucleotide” are used interchangeably herein, and refer to both RNA and DNA molecules, including nucleic acid molecules including cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of a lentiviral vector, which may be transduced into a cell. Polynucleotides useful in the methods of the disclosure may comprise natural nucleic acid sequences and variants thereof, artificial nucleic acid sequences, or a combination of such sequences.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

In some embodiments, the nucleic acid molecules of the disclosure are recombinant, e.g. nucleic acid molecules that have been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

As used herein, the term “construct” is intended to mean any recombinant nucleic acid molecule. In embodiments, a construct includes an expression cassette, plasmid, cosmid, virus, autonomously replicating polynucleotide molecule, phage, or linear or circular, single-stranded or double-stranded, DNA or RNA polynucleotide molecule. A construct may be derived from any source, capable of genomic integration or autonomous replication, including a nucleic acid molecule where one or more nucleic acid sequences has been linked in a functionally operative manner, e.g., operably linked.

The terms “operably linked” or “functionally linked”, are interchangeable and denote a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is functional link that allows for expression of the polynucleotide of interest. In this sense, the term “operably linked” refers to the positioning of a regulatory region (e.g. a promoter) and a coding sequence (e.g. polynucleotide encoding a gene editing agent, etc.) to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, operably linked elements may be contiguous or non-contiguous. In addition, in the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, regions, or domains of the engineered antibodies disclosed herein may be operably linked to retain proper folding, processing, targeting, expression, binding, and other functional properties of the engineered antibodies in the cell. Unless stated otherwise, various regions, domains, and segments of the engineered antibodies of the disclosure are operably linked to each other. Operably linked regions, domains, and segments of the engineered antibodies of the disclosure may be contiguous or non-contiguous (e.g., linked to one another through a linker).

The term “recombination” as used herein refers to a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (e.g., the one that experienced the double-strand break), and leads to the transfer of genetic information from the donor to the target. Homology-directed repair may result in an alteration of the sequence of the target molecule (e.g. insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA.

The term “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., tEGFR) in other proteins with different numbering systems. For example, by performing a simple sequence alignment with a protein (e.g., tEGFR) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the residue to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI or UniProt sequence reference. In other embodiments, the protein is the protein as identified by its NCBI or UniProt sequence reference, homolog or functional fragment thereof.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The term “CCR5 protein” or “CCR5” as used herein includes any of the recombinant or naturally-occurring forms of C—C chemokine receptor type 5, also known as C—C CKR-5, or variants or homologs thereof that maintain CCR5 activity (e.g. within at least 50%, 80%, 90%, 95%, 96%, 97%, 98%, 99% or 100% activity compared to CCR5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCR5 protein. In embodiments, the CCR5 protein is substantially identical to the protein identified by the UniProt reference number P51681 or a variant or homolog having substantial identity thereto.

As used herein, the terms “genetic modification”, “gene modification”, “gene editing”, “genetic editing”, “genome editing”, “genome engineering” or the like refer to a type of genetic engineering in which DNA is inserted, deleted, modified or replaced at one or more specified locations in the genome of a cell. Unlike early genetic engineering techniques that randomly insert genetic material into a host genome, genome editing targets the insertions to site specific locations. In embodiments, a step in gene editing is creation of a double stranded break at a specific point within a gene or genome. Examples of gene editing tools such as nucleases that accomplish this step include but are not limited to Zinc finger nucleases (ZFNs), transcription activator like effector nucleases (TALEN), meganucleases, and clustered regularly interspaced short palindromic repeats system (CRISPR/Cas).

As used herein, the term “gene knockout” or “KO” refers to a genetic technique in which one of an organism's genes is made totally or partially inoperative. A knockout can be heterozygous and homozygous KOs. In the former, only one of two gene copies (alleles) is knocked out, in the latter both are knocked out. In embodiments, near complete loss of target gene expression at the population level may be accomplished, which mitigates the need for selection steps.

The term “loss of function” as used herein refers to a mutation within a gene or deletion of a portion or the entirety or the gene that results in loss of function of the gene product or protein encoded by the gene. In embodiments, loss of function refers to decreasing or inhibiting the activity of the gene product or protein by 90%, 80%, 70%, 60%, 50%, 40%, 30%, 20%, or 10% compared to the activity of the gene product or protein in the absence of the mutation or gene deletion. In embodiments, loss of function is decreasing or inhibiting the activity of the gene product or gene by 1.5-fold, 2-fold, 3-fold, 4-fold, 5-fold, 10-fold, or more in comparison to the activity of the gene product or gene in the absence of the mutation or gene deletion.

As used herein, the term “gene editing agent” refers to components required for gene editing tools and may include enzymes, riboproteins, solutions, co-factors and the like. For example, gene editing agents include one or more components required for Zinc finger nucleases (ZFNs), transcription activator like effector nucleases (TALEN), meganucleases, and clustered regularly interspaced short palindromic repeats system (CRISPR/Cas) gene editing.

The term “site-specific modifying enzyme” or “RNA-binding site-specific modifying enzyme” as used herein refers to a polypeptide that binds RNA and is targeted to a specific DNA sequence, for example a Cas9 polypeptide. A site-specific modifying enzyme as described herein is targeted to a specific DNA sequence by the RNA molecule to which it is bound. The RNA molecule includes a sequence that binds, hybridizes to, or is complementary to a target sequence within the target DNA, thus targeting the bound polypeptide to a specific location within the target DNA (the target sequence).

“Nuclease” and “endonuclease” are used interchangeably herein to mean an enzyme which possesses endonucleolytic catalytic activity for polynucleotide cleavage. The term includes site-specific endonucleases such as, designer zinc fingers, transcription activator-like effectors (TALEs), homing meganucleases, and site-specific endonucleases of clustered, regularly interspaced, short palindromic repeat (CRISPR) systems such as, e.g., Cas proteins.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

As used herein, the term “CRISPR” or “clustered regularly interspaced short palindromic repeats” is used in accordance with its plain ordinary meaning and refers to a genetic element that bacteria use as a type of acquired immunity to protect against viruses. CRISPR includes short sequences that originate from viral genomes and have been incorporated into the bacterial genome. Cas (CRISPR associated proteins) process these sequences and cut matching viral DNA sequences. Thus, CRISPR sequences function as a guide for Cas to recognize and cleave DNA that are at least partially complementary to the CRISPR sequence. By introducing plasmids including Cas genes and specifically constructed CRISPRs into eukaryotic cells, the eukaryotic genome can be cut at any desired position. The term “Class II CRISPR endonuclease” refers to endonucleases that have similar endonuclease activity as Cas9 and participate in a Class II CRISPR system. An example Class II CRISPR system is the type II CRISPR locus from Streptococcuspyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system Both type II and type V systems are included in Class II of the CRISPR-Cas system. In embodiments, the CRISPR system may be any such system, including without limitation, Cas9, SpCas9, SaCas9, NmCas9, StlCas9, FnCas9, Cas12a (e.g. FnCpf1, AsCpfl, LbCpfl), Mad7, CasX, CasY, Cas13a, C2c1, C2c2, C2c3, LshC2c2, Cas14, dSpCas9-FokI, Split-SpCas9, SpCas9-nickase. Additional CRISPR systems are known, for example as described in Komor et al., Nature (2016) 533(7603):420-4, which is incorporated herein by reference in its entirety.

A “guide RNA” or “gRNA” as provided herein refers to an RNA sequence having sufficient complementarity with a target polynucleotide sequence to hybridize with the target sequence and direct sequence-specific binding of a CRISPR complex to the target sequence. For example, a gRNA can direct Cas to the target polynucleotide. In embodiments, the gRNA includes the crRNA and the tracrRNA. For example, the gRNA can include the crRNA and tracrRNA hybridized by base pairing. Thus, in embodiments, the two RNA can be encoded separately by a crRNA and tracrRNA as 2 RNA molecules which then form an RNA/RNA complex due to complementary base pairing between the crRNA and tracrRNA. In aspects, the degree of complementarity between a guide RNA sequence and its corresponding target sequence, when optimally aligned using a suitable alignment algorithm, is about 50%, 60%, 75%, 80%, 85%, 90%, 95%, 97.5%, 99%, or more. In aspects, the degree of complementarity between a guide RNA sequence and its corresponding target sequence, when optimally aligned using a suitable alignment algorithm, is at least about 50%, 60%, 75%, 80%, 85%, 90%, 95%, 97.5%, 99%.

The terms “sgRNA,” “single guide RNA,” and “single guide RNA sequence” are used interchangeably and refer to an RNA sequence including the crRNA sequence and the tracrRNA sequence. For example, the sgRNA can be a single RNA sequence including the crRNA and tracrRNA. For example, the sgRNA can be a fusion sequence including the crRNA and tracrRNA. In embodiments, the sgRNA is synthesized in vitro. In embodiments, the sgRNA is made in vivo from a DNA sequence encoding the sgRNA.

As used herein, the term “Cas9” or “CRISPR-associated protein 9” is used in accordance with its plain ordinary meaning and refers to an enzyme that uses CRISPR sequences as a guide to recognize and cleave specific strands of DNA that are at least partically complementary to the CRISPR sequence. Cas9 enzymes together with CRISPR sequences form the basis of a technology known as CRISPR-Cas9 that can be used to edit genes within organisms. This editing process has a wide variety of applications including basic biological research, development of biotechnology products, and treatment of diseases.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number A0Q7Q2 or a variant or homolog having substantial identity thereto.

A “CRISPR-associated endoribonuclease Cas13a,” “Cas13a,” “Cas13” or “Cas13 protein” as referred to herein includes any of the recombinant or naturally-occurring forms of the Cas13 endoribonuclease or variants or homologs thereof that maintain Cas13 endoribonuclease enzyme activity (e.g. within at least 50%, 80%, 90%, 95%, 96%, 97%, 98%, 99% or 100% activity compared to Cas13). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13 protein. In aspects, the Cas13 protein is substantially identical to the protein identified by the UniProt reference number PODPB8 or a variant or homolog having substantial identity thereto.

As used herein, “Cascade” refers to the complex of Cas proteins associated with an RNA sequence including the CRISPR sequence. For example, Cascade may include one or more Cas proteins (e.g. Cas9), and cleave target DNA as directed by the CRISPR sequence. In other examples, the Cascade complex may display the CRISPR RNA and recruit Cas proteins (e.g. Cas3) to cleave the target DNA.

An “argonaut endonuclease,” “argonaut,” “protein argonaute-2” or “argonaut protein” as referred to herein includes any of the recombinant or naturally-occurring forms of the argonaut endonuclease or variants or homologs thereof that maintain argonaut endonuclease enzyme activity (e.g. within at least 50%, 80%, 90%, 95%, 96%, 97%, 98%, 99% or 100% activity compared to argonaut). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring argonaut protein. In aspects, the argonaut protein is substantially identical to the protein identified by the UniProt reference number Q9UKV8 or a variant or homolog having substantial identity thereto.

As used herein, “TALEN” or “transcription activator-like effector nuclease” refers to restriction enzymes generated by attaching a DNA binding domain (e.g. a TAL effector DNA-binding domain) to a nuclease (e.g. FokI). TALEN typically includes a naturally occurring DNA-binding domain, which include multiple modules, termed TALs or TALEs. Thus, the TALs, which include variable diresidues, confer DNA binding specificity.

A “ribonucleoprotein complex,” or “ribonucleoprotein particle” as provided herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs). The ribonucleoprotein may be an enzyme. In embodiments, the ribonucleoprotein is an endonuclease. Thus, in embodiments, the ribonucleoprotein complex includes an endonuclease and a ribonucleic acid. In embodiments, the endonuclease is a CRISPR associated protein 9.

By “cleavage” it is meant the breakage of the covalent backbone of a polynucleotide or polypeptide molecule. For polynucleotides, cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In some embodiments, a complex including a guide RNA and a site-specific modifying enzyme is used for targeted double-stranded DNA cleavage. Polypeptides can be cleaved by enzymes including proteases (e.g. furin). For example, cleavage can occur by breaking of the peptide bond within proteins by hydrolysis, thereby cleaving the protein into shorter peptide sequences or single amino acid residues.

By “cleavage domain” or “active domain” or “nuclease domain” of a nuclease it is meant the polypeptide sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “transcription start site” and “transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be, e.g., about 100 to about 1000 base pairs in length.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “vector” or “expression vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

An expression vector can be introduced into prokaryotic or eukaryotic cells via conventional transformation or transfection techniques. Suitable methods for transforming or transfecting host cells can be found in Sambrook et al. (2001, supra) and other standard molecular biology laboratory manuals.

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered.

In selecting an expression control sequence, a variety of factors should also be considered. These include, for example, the relative strength of the sequence, its controllability, and its compatibility with the actual DNA sequence encoding the subject polypeptide, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, including the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents. A “memory T-cell” is a T-cell that has previously encountered and responded to its cognate antigen during prior infection, encounter with cancer or previous vaccination. At a second encounter with its cognate antigen memory T-cells can reproduce (divide) to mount a faster and stronger immune response than the first time the immune system responded to the pathogen. A “regulatory T-cell” or “suppressor T-cell” is a lymphocyte which modulates the immune system, maintains tolerance to self-antigens, and prevents autoimmune disease.

A “stem cell” is a cell characterized by the ability of self-renewal through mitotic cell division and the potential to differentiate into a tissue or an organ. The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. A “hematopoietic stem cell” as provided herein refers to a somatic stem cell that is able to give rise to all blood cells. A hematopoietic stem cell has the capacity to differentiate into cells of the myeloid lineage (i.e. erythrocytes, mast cells, basophils, neutrophils, eosinophils, monocytes and macrophages) and the lymphoid lineage (i.e. natural killer cells, T cells and B cells).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a viral infection (e.g. HIV). The disease may be thalassemia. The disease may be sickle cell anemia. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

The term “red blood cell disease” refers to a disease affecting a red blood cell. Non-limiting examples of red blood cell diseases include anemia, sickle cell disease, acute lymphoblastic leukemia, hemolytic anemia, aplastic anemia, thalassemia, polycythemia, myelodysplastic syndrome, polycythemia vera, iron-deficiency anemia, autoimmune hemolytic anemia, sphercytosis, hereditary spherocytosis, megaloblastic anemia, glucose-6-phosphate dehydrogenase deficiency, normocytic anemia, paroxysmal nocturnal hemoglobinuria, hypochromic anemia, macrocytic anemia, pyruvate kinase deficiency, hereditary stomatocytosis, microcytosis, microcytic anemia, macrocytosis and hereditary elliptocytosis.

In some instances, “disease” or “condition” refer to “hematological disease. “A hematological disease refers to a disease affecting a hematologic cell. In some instances, the hematological disease is a non-cancerous (i.e. non-malignant) hematological disease. Non-cancerous hematological diseases as provided herein include any disease, disorder or condition related to hematologic cells that is not cancer. Examples of non-cancerous hematological diseases, disorders, or conditions include, but are not limited to hemoglobinopathies including sickle-cell disease, thalassemia, methemoglobinemia; anemias including iron deficiency anemia, folate deficiency, hemolytic anemias, megaloblastic anemia, vitamin B12 deficiency, pernicious anemia, immune mediated hemolytic anemia, drug-induced immune mediated hemolytic anemia (e.g. due to high dose of penicillin, methyldopa), hemoglobinopathies, paroxysmal nocturnal hemoglobinuria, and microangiopathic hemolytic anemia; disease characterized by decreased numbers of blood cells (e.g. erythrocytes, lymphocytes, myeloid cells) including myelodysplastic syndrome, myelofibrosis, neutropenia, agranulocytosis, Glanzmann's thrombasthenia, thrombocytopenia, idiopathic thrombocytopenic purpura, thrombotic thrombocytopenic purpura, and heparin-induced thrombocytopenia; myeloproliferative disorders including polycythemia vera, erythrocytosis, leukocytosis, and thrombocytosis; coagulopathies including thrombocytosis, recurrent thrombosis, disseminated intravascular coagulation, hemophilia, Von Willebrand disease, disseminated intravascular coagulation, protein S deficiency, and antiphospholipid syndrome.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. For example, administration may be infusion of genetically modified cells into a subject in need thereof. In embodiments, the administering does not include administration of any active agent other than the recited active agent.

“Co-administer” it is meant that a composition described herein is administered at the same time, just prior to, or just after the administration of one or more additional therapies. The compositions and compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). For example, the genetically modified cells provided herein including embodiments thereof may be co-administered with a small molecule therapeutic.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

### Nucleic Acid Constructs

The compositions provided herein include nucleic acid constructs including polynucleotides encoding tEGFR, a gene editing agent, and a linker peptide. The nucleic acid constructs can be delivered into the cell for genetic modification and sorting of the cell. Expression of the gene editing agent allows for gene modification, and expression of tEGFR allows for identification and selection of the genetically modified cell. In embodiments, the polynucleotide linker peptide is cleavable and is located between the tEGFR and the gene editing agent. In instances, cleavage of the linker peptide allows for shuttling of the gene editing agent to the target gene, and the tEGFR to the cell surface. Thus, in an aspect is provided a nucleic acid construct including a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent.

In embodiments, the nucleic acid further includes a polynucleotide encoding a cleavage site. In embodiments, the cleavage site is a furin cleavage site. In embodiments, the furin cleavage site sequence includes GGAGAGAGRSRAKRSVGGAGAGAG (SEQ ID NO: 72). In embodiments, the furin cleavage site includes the amino acid sequence: RXXR, wherein X is a naturally occurring amino acid residue. In embodiments, the furin cleavage site includes the amino acid sequence: RX[R/K]R. In embodiments, the furin cleavage site includes the amino acid sequence: RRKR or RKRR. In embodiments, the furin cleavage site includes the amino acid sequence RRKR. In embodiments, the furin cleavage site includes the amino acid sequence RKRR.

In embodiments, the linker peptide is a cleavable linker. In embodiments, the cleavable linker is a self-cleaving linker. In embodiments, the linker peptide includes a self-cleaving peptide. In embodiments, the self-cleaving peptide includes a ribosomal skip sequence. As used herein, “ribosomal skip sequence”, also known as “self-cleaving peptidyl sequence”, refers to a class of peptide sequences that can induce ribosomal skipping during translation. Thus, in instances, presence of a ribosomal skep sequence results in the generation of multiple peptides originally encoded by a single mRNA. In embodiments, the ribosomal skip sequence includes a T2A sequence, E2A sequence, P2A sequence, or F2A sequence. In embodiments, the ribosomal skip sequence is a T2A sequence, E2A sequence, P2A sequence, or F2A sequence. In embodiments, the ribosomal skip sequence is a T2A sequence. In embodiments, the ribosomal skip sequence is a E2A sequence. In embodiments, the ribosomal skip sequence is a P2A sequence. In embodiments, the ribosomal skip sequence is a F2A sequence. In embodiments, the T2A sequence includes GRGSLLTCGDVEENPGP (SEQ ID NO:65). In embodiments, the P2A ribosomal skip sequence includes ATNFSLLKQAGDVEENPGP (SEQ ID NO:66). In embodiments, the E2A ribosomal skip sequence includes QCTNYALLKLAGDVESNPGP (SEQ ID NO:67) In embodiments, the F2A ribosomal skip sequence includes VKQTLNFDLLKLAGDVESNPGP (SEQ ID NO:68).

In embodiments, the linker peptide is between 2 amino acid residues and 40 amino acids in length. In embodiments, the linker peptide is between 2 amino acid residues and 4 amino acids in length. In embodiments, the linker peptide is between 2 amino acid residues and 6 amino acids in length. In embodiments, the linker peptide is between 2 amino acid residues and 8 amino acids in length. In embodiments, the linker peptide is between 2 amino acid residues and 10 amino acids in length. In embodiments, the linker peptide is between 2 amino acid residues and 12 amino acids in length. In embodiments, the linker peptide is between 2 amino acid residues and 14 amino acids in length. In embodiments, the linker peptide is between 2 amino acid residues and 16 amino acids in length. In embodiments, the linker peptide is between 2 amino acid residues and 18 amino acids in length. In embodiments, the linker peptide is between 2 amino acid residues and 20 amino acids in length. In embodiments, the linker peptide is between 2 amino acid residues and 22 amino acids in length. In embodiments, the linker peptide is between 2 amino acid residues and 24 amino acids in length. In embodiments, the linker peptide is between 2 amino acid residues and 26 amino acids in length. In embodiments, the linker peptide is between 2 amino acid residues and 28 amino acids in length. In embodiments, the linker peptide is between 2 amino acid residues and 30 amino acids in length. In embodiments, the linker peptide is between 2 amino acid residues and 32 amino acids in length. In embodiments, the linker peptide is between 2 amino acid residues and 34 amino acids in length. In embodiments, the linker peptide is between 2 amino acid residues and 36 amino acids in length. In embodiments, the linker peptide is between 2 amino acid residues and 38 amino acids in length. In embodiments, the linker peptide is between 2 amino acid residues and 40 amino acids in length.

In embodiments, the linker peptide is between 2 amino acid residues and 38 amino acids in length. In embodiments, the linker peptide is between 2 amino acid residues and 36 amino acids in length. In embodiments, the linker peptide is between 2 amino acid residues and 34 amino acids in length. In embodiments, the linker peptide is between 2 amino acid residues and 32 amino acids in length. In embodiments, the linker peptide is between 2 amino acid residues and 30 amino acids in length. In embodiments, the linker peptide is between 2 amino acid residues and 28 amino acids in length. In embodiments, the linker peptide is between 2 amino acid residues and 26 amino acids in length. In embodiments, the linker peptide is between 2 amino acid residues and 24 amino acids in length. In embodiments, the linker peptide is between 2 amino acid residues and 22 amino acids in length. In embodiments, the linker peptide is between 2 amino acid residues and 20 amino acids in length. In embodiments, the linker peptide is between 2 amino acid residues and 18 amino acids in length. In embodiments, the linker peptide is between 2 amino acid residues and 16 amino acids in length. In embodiments, the linker peptide is between 2 amino acid residues and 14 amino acids in length. In embodiments, the linker peptide is between 2 amino acid residues and 12 amino acids in length. In embodiments, the linker peptide is between 2 amino acid residues and 10 amino acids in length. In embodiments, the linker peptide is between 2 amino acid residues and 8 amino acids in length. In embodiments, the linker peptide is between 2 amino acid residues and 6 amino acids in length. In embodiments, the linker peptide is between 2 amino acid residues and 4 amino acids in length. In embodiments, the linker peptide is between 2 amino acid residues, 4 amino acid residues, 6 amino acid residues, 8 amino acid residues, 10 amino acid residues, 12 amino acid residues, 14 amino acid residues, 16 amino acid residues, 18 amino acid residues, 20 amino acid residues, 22 amino acid residues, 24 amino acid residues, 26 amino acid residues, 28 amino acid residues, 30 amino acid residues, 32 amino acid residues, 34 amino acid residues, 36 amino acid residues, 38 amino acid residues, or 40 amino acid residues in length.

It is contemplated that a nucleic acid construct encoding a plurality of cleavage sequences is effective for efficiently cleaving the polypeptide encoded by the construct. Thus, for the construct provided herein, in embodiments, the construct includes a peptide linker and a cleavage site. In embodiments, the peptide linker includes a self-cleaving peptide, wherein the self-cleaving peptide is a ribosomal skip sequence. In embodiments, the cleavage site is a furin cleavage site. Thus, in embodiments, the construct includes a sequence including a furin cleavage site adjacent to a ribosomal skip sequence, referred to herein as a “furin-ribosomal skip site”. In embodiments, the furin-ribosomal skip site includes a linker sequence (e.g. GSG) between the furin cleavage site and the ribosomal skip sequence. In embodiments, the furin-ribosomal skip site is encoded by a nucleic acid sequence of SEQ ID NO.:3. In embodiments, the furin-ribosomal skip site is encoded by a nucleic acid sequence of SEQ ID NO.:11. In embodiments, the furin-ribosomal skip site is encoded by a nucleic acid sequence of SEQ ID NO.:19. In embodiments, the furin-ribosomal skip site is encoded by a nucleic acid sequence of SEQ ID NO.:27. In embodiments, the furin-ribosomal skip site is encoded by a nucleic acid sequence of SEQ ID NO.:35. In embodiments, the furin-ribosomal skip site is encoded by a nucleic acid sequence of SEQ ID NO.:43. In embodiments, the furin-ribosomal skip site is encoded by a nucleic acid sequence of SEQ ID NO.:51. In embodiments, the furin-ribosomal skip site is encoded by a nucleic acid sequence of SEQ ID NO.:59.

In embodiments, the furin-ribosomal skip site includes the amino acid sequence of SEQ ID NO.:7. In embodiments, the furin-ribosomal skip site includes the amino acid sequence of SEQ ID NO.:15. In embodiments, the furin-ribosomal skip site includes the amino acid sequence of SEQ ID NO.:23. In embodiments, the furin-ribosomal skip site includes the amino acid sequence of SEQ ID NO.:31. In embodiments, the furin-ribosomal skip site includes the amino acid sequence of SEQ ID NO.:39. In embodiments, the furin-ribosomal skip site includes the amino acid sequence of SEQ ID NO.:47. In embodiments, the furin-ribosomal skip site includes the amino acid sequence of SEQ ID NO.:55. In embodiments, the furin-ribosomal skip site includes the amino acid sequence of SEQ ID NO.:63.

In embodiments, the nucleic acid construct further includes a promoter operably linked to the polynucleotide encoding the gene editing agent, the polynucleotide encoding the linker peptide, and the polynucleotide encoding the tEGFR. In embodiments, the promoter is a CMV promoter, a EF1α promoter, a PGK promoter, a CAG.SV40 promoter, or a Ubc promoter. In embodiments, the promoter is a CMV promoter. In embodiments, the promoter is a EF1α promoter. In embodiments, the promoter is a PGK promoter. In embodiments, the promoter is a CAG.SV40 promoter. In embodiments, the promoter is a Ubc promoter.

For the nucleic acid construct provided herein, in embodiments, the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease. In embodiments, the gene editing agent is a meganuclease. In embodiments, the gene editing agent is a CRISPR protein. In embodiments, the gene editing agent is a TALEN. In embodiments, the gene editing agent is a zinc finger nuclease. In embodiments, the gene editing agent is a MegaTAL. In embodiments, the gene editing agent is an Argonaute endonuclease. In embodiments, gene editing agent includes an RNA-guided nuclease. In embodiments, the RNA-guided nuclease includes a Cas protein or variant thereof. In embodiments, one or more gene editing agents may be expressly excluded.

In embodiments, the polynucleotide encoding the tEGFR is 3′ of the polynucleotide encoding the linker peptide, and the polynucleotide encoding the cleavage site is 3′ of the polynucleotide encoding the gene editing agent. Thus, in embodiments, the nucleic acid construct includes, from the 5′ direction to the 3′ direction, the polynucleotide encoding a gene editing agent, the polynucleotide encoding a linker peptide, and a polynucleotide encoding a truncated tEGFR. In embodiments, the nucleic acid construct includes, from the 5′ direction to the 3′ direction, the polynucleotide encoding a gene editing agent, the polynucleotide encoding a cleavage site, the polynucleotide encoding a linker peptide, and a polynucleotide encoding a truncated tEGFR. In embodiments, the nucleic acid construct includes, from the 5′ direction to the 3′ direction, the polynucleotide encoding a gene editing agent, the polynucleotide encoding a cleavage site, the polynucleotide encoding a GSG linker, the polynucleotide encoding a linker peptide, and a polynucleotide encoding a truncated tEGFR.

In embodiments, the polynucleotide encoding the gene editing agent is 3′ of the polynucleotide encoding the linker peptide, and the polynucleotide encoding the linker peptide is 3′ of the polynucleotide encoding the tEGFR. Thus, in embodiments, the nucleic acid construct includes, from the 5′ direction to the 3′ direction, the polynucleotide encoding a truncated tEGFR, the polynucleotide encoding a linker peptide, and the polynucleotide encoding a gene editing agent. In embodiments, the nucleic acid construct includes, from the 5′ direction to the 3′ direction, the polynucleotide encoding a truncated tEGFR, the polynucleotide encoding a cleavage site, the polynucleotide encoding a linker peptide, and the polynucleotide encoding a gene editing agent. In embodiments, the nucleic acid construct includes, from the 5′ direction to the 3′ direction, the polynucleotide encoding a truncated tEGFR, the polynucleotide encoding a cleavage site, the polynucleotide encoding a GSG linker, the polynucleotide encoding a linker peptide, and the polynucleotide encoding a gene editing agent.

In embodiments, the nucleic acid includes a nucleotide encoding a GSG linker. In embodiments, the GSG linker is 3′ of the polynucleotide encoding the linker peptide. In instances, the polynucleotide encoding GSG linker is located between the polynucleotide encoding a cleavage site and the polynucleotide encoding a linker peptide.

In embodiments, the nucleic acid construct is DNA. In embodiments, the nucleic acid construct is RNA. As would be understood by one having skill in the art, in embodiments where the nucleic acid is RNA, one or more of the thymine bases as represented in the sequence listing for any nucleotide sequence provided therein may be replaced by uracil. In embodiments, all of the thymine bases as represented in the sequence listing may be replaced by uracil.

In embodiments, the tEGFR is encoded by a nucleic acid sequence of any one of SEQ ID NO:4, SEQ ID NO.:12, SEQ ID NO.:20, SEQ ID NO.:36, SEQ ID NO.:52, or SEQ ID NO.:60, or a nucleic acid sequence having at least about 85% identity therewith. In embodiments, the tEGFR includes an amino acid sequence of any one of SEQ ID NO.: 8, SEQ ID NO.:16, SEQ ID NO.:24, SEQ ID NO.:40, SEQ ID NO.:56, SEQ ID NO.: 64, or an amino acid sequence having at least about 85% identity therewith. tEGFR is described in U.S. Pat. No. 8,802,374, which is incorporated herein in its entirety for all sequences, methods, constructs, and all other teachings therein.

In embodiments, the gene editing agent is encoded by a nucleic acid sequence of SEQ ID NO.:2. In embodiments, the gene editing agent is encoded by a nucleic acid sequence of SEQ ID NO.: 10. In embodiments, the gene editing agent is encoded by a nucleic acid sequence of SEQ ID NO.: 18. In embodiments, the gene editing agent is encoded by a nucleic acid sequence of SEQ ID NO.:26. In embodiments, the gene editing agent is encoded by a nucleic acid sequence of SEQ ID NO.:34. In embodiments, the gene editing agent is encoded by a nucleic acid sequence of SEQ ID NO.:42. In embodiments, the gene editing agent is encoded by a nucleic acid sequence of SEQ ID NO.:50. In embodiments, the gene editing agent is encoded by a nucleic acid sequence of SEQ ID NO.:58. In embodiments, the gene editing agent is encoded by a nucleic acid sequence having at least 85% identity to any one of SEQ ID NO.:2, SEQ ID NO.:10, SEQ ID NO.:18, SEQ ID NO.:26, SEQ ID NO.:34, SEQ ID NO.: 42, SEQ ID NO.:50, or SEQ ID NO.:58.

In embodiments, the gene editing agent comprises an amino acid sequence of SEQ ID NO.: 6. In embodiments, the gene editing agent comprises an amino acid sequence of SEQ ID NO.: 14. In embodiments, the gene editing agent comprises an amino acid sequence of SEQ ID NO.:22. In embodiments, the gene editing agent comprises an amino acid sequence of SEQ ID NO.:30. In embodiments, the gene editing comprises an amino acid sequence of SEQ ID NO.:38. In embodiments, the gene editing agent comprises an amino acid sequence of SEQ ID NO.:46. In embodiments, the gene editing agent comprises an amino acid sequence of SEQ ID NO.:54. In embodiments, the gene editing agent comprises an amino acid sequence of SEQ ID NO.:62. In embodiments, the gene editing agent comprises an amino acid sequence having at least 85% identity to any one of SEQ ID NO.:6, SEQ ID NO.:14, SEQ ID NO.:22, SEQ ID NO.:30, SEQ ID NO.:38, SEQ ID NO.:46, SEQ ID NO.: 54, or SEQ ID NO.: 62.

In embodiments, the polypeptide encoded by the nucleic acid construct comprises the amino acid sequence of SEQ ID NO.:69. In embodiments, the polypeptide encoded by the nucleic acid construct comprises the amino acid sequence of SEQ ID NO.:70. In embodiments, the polypeptide encoded by the nucleic acid construct comprises the amino acid sequence of SEQ ID NO.:71.

In embodiments, the nucleic acid construct includes the nucleic acid sequence of SEQ ID NO.:1. In embodiments, the nucleic acid construct includes a nucleic acid sequence having at least 85% identity to the nucleic acid sequence of SEQ ID NO.:1. In embodiments, the nucleic acid construct includes a nucleic acid sequence having at least 90%, 95%, 96%, 97%, 98%, or 99% identity to the nucleic acid sequence of SEQ ID NO.:1.

In embodiments, the nucleic acid construct includes the nucleic acid sequence of SEQ ID NO.:9. In embodiments, the nucleic acid construct includes a nucleic acid sequence having at least 85% identity to the nucleic acid sequence of SEQ ID NO.:9. In embodiments, the nucleic acid construct includes a nucleic acid sequence having at least 90%, 95%, 96%, 97%, 98%, or 99% identity to the nucleic acid sequence of SEQ ID NO.:9.

In embodiments, the nucleic acid construct includes the nucleic acid sequence of SEQ ID NO.:17. In embodiments, the nucleic acid construct includes a nucleic acid sequence having at least 85% identity to the nucleic acid sequence of SEQ ID NO.:17. In embodiments, the nucleic acid construct includes a nucleic acid sequence having at least 90%, 95%, 96%, 97%, 98%, or 99% identity to the nucleic acid sequence of SEQ ID NO.:17.

In embodiments, the nucleic acid construct includes the nucleic acid sequence of SEQ ID NO.:33. In embodiments, the nucleic acid construct includes a nucleic acid sequence having at least 85% identity to the nucleic acid sequence of SEQ ID NO.:33. In embodiments, the nucleic acid construct includes a nucleic acid sequence having at least 90%, 95%, 96%, 97%, 98%, or 99% identity to the nucleic acid sequence of SEQ ID NO.:33.

In embodiments, the nucleic acid construct includes the nucleic acid sequence of SEQ ID NO.:49. In embodiments, the nucleic acid construct includes a nucleic acid sequence having at least 85% identity to the nucleic acid sequence of SEQ ID NO.:49. In embodiments, the nucleic acid construct includes a nucleic acid sequence having at least 90%, 95%, 96%, 97%, 98%, or 99% identity to the nucleic acid sequence of SEQ ID NO.:49.

In embodiments, the nucleic acid construct includes the nucleic acid sequence of SEQ ID NO.:57. In embodiments, the nucleic acid construct includes a nucleic acid sequence having at least 85% identity to the nucleic acid sequence of SEQ ID NO.:57. In embodiments, the nucleic acid construct includes a nucleic acid sequence having at least 90%, 95%, 96%, 97%, 98%, or 99% identity to the nucleic acid sequence of SEQ ID NO.:57.

In embodiments, the nucleic acid construct encodes a protein, peptide, and/or polypeptide. In embodiments, the protein, peptide, and/or polypeptide includes the amino acid sequence of SEQ ID NO.:5. In embodiments, the protein, peptide, and/or polypeptide includes an amino acid sequence having at least 85% identity to the amino acid sequence of SEQ ID NO.:5. In embodiments, the protein, peptide, and/or polypeptide includes an amino acid sequence having at least 90%, 95%, 96%, 97%, 98%, or 99% identity to the amino acid sequence of SEQ ID NO.:5.

In embodiments, the nucleic acid construct encodes a protein, peptide, and/or polypeptide. In embodiments, the protein, peptide, and/or polypeptide includes the amino acid sequence of SEQ ID NO.:13. In embodiments, the protein, peptide, and/or polypeptide includes an amino acid sequence having at least 85% identity to the amino acid sequence of SEQ ID NO.:13. In embodiments, the protein, peptide, and/or polypeptide includes an amino acid sequence having at least 90%, 95%, 96%, 97%, 98%, or 99% identity to the amino acid sequence of SEQ ID NO.:13.

In embodiments, the nucleic acid construct encodes a protein, peptide, and/or polypeptide. In embodiments, the protein, peptide, and/or polypeptide includes the amino acid sequence of SEQ ID NO.:21. In embodiments, the protein, peptide, and/or polypeptide includes an amino acid sequence having at least 85% identity to the amino acid sequence of SEQ ID NO.:21. In embodiments, the protein, peptide, and/or polypeptide includes an amino acid sequence having at least 90%, 95%, 96%, 97%, 98%, or 99% identity to the amino acid sequence of SEQ ID NO.:21.

In embodiments, the nucleic acid construct encodes a protein, peptide, and/or polypeptide. In embodiments, the protein, peptide, and/or polypeptide includes the amino acid sequence of SEQ ID NO.:37. In embodiments, the protein, peptide, and/or polypeptide includes an amino acid sequence having at least 85% identity to the amino acid sequence of SEQ ID NO.:37. In embodiments, the protein, peptide, and/or polypeptide includes an amino acid sequence having at least 90%, 95%, 96%, 97%, 98%, or 99% identity to the amino acid sequence of SEQ ID NO.:37.

In embodiments, the nucleic acid construct encodes a protein, peptide, and/or polypeptide. In embodiments, the protein, peptide, and/or polypeptide includes the amino acid sequence of SEQ ID NO.:53. In embodiments, the protein, peptide, and/or polypeptide includes an amino acid sequence having at least 85% identity to the amino acid sequence of SEQ ID NO.:53. In embodiments, the protein, peptide, and/or polypeptide includes an amino acid sequence having at least 90%, 95%, 96%, 97%, 98%, or 99% identity to the amino acid sequence of SEQ ID NO.:53.

In embodiments, the nucleic acid construct encodes a protein, peptide, and/or polypeptide. In embodiments, the protein, peptide, and/or polypeptide includes the amino acid sequence of SEQ ID NO.:61. In embodiments, the protein, peptide, and/or polypeptide includes an amino acid sequence having at least 85% identity to the amino acid sequence of SEQ ID NO.:61. In embodiments, the protein, peptide, and/or polypeptide includes an amino acid sequence having at least 90%, 95%, 96%, 97%, 98%, or 99% identity to the amino acid sequence of SEQ ID NO.:61.

The nucleic acid construct may include any polynucleotide as described herein including embodiments thereof.

### Expression Vectors

The nucleic acid construct provided herein including embodiments thereof, may be delivered to a cell in a variety of methods known in the art. The nucleic acid construct may be RNA or DNA delivered to cells as a modified or unmodified RNA or plasmid DNA. In embodiments, the nucleic acid construct provided herein including embodiments thereof may be delivered as a messenger RNA. The nucleic acid construct may be delivered by transfection, lipid nanoparticle, virus like particle (VLP) or virus.

It is further contemplated that the nucleic acid construct provided herein, including embodiments thereof, may be included in an expression vector. Therefore, in an aspect is provided an expression vector including the nucleic acid construct provided herein including embodiments thereof. In embodiments, the vector is a viral vector or a plasmid. In embodiments, the vector is a viral vector. In embodiments, the vector is a plasmid.

### Cells

The nucleic acid compositions described herein may be incorporated into a cell. Inside the cell, the proteins encoded by the nucleic acids as described herein, including embodiments and aspects thereof, may be expressed and perform genetic modifications. Thus, in an aspect is provided a cell including the nucleic acid construct provided herein including embodiments thereof. In another aspect is provided a cell including the expression vector provided herein including embodiments thereof. In an aspect is provided a population of cells comprising a nucleic acid construct as described herein including embodiments thereof.

In embodiments, the cell is a mammalian cell. In embodiments, the cell is a human cell. In embodiments, the cell is an immune cell. In embodiments, the cell is a T cell. In embodiments, the cell does not express endogenous EGFR. In embodiments, the mammalian cell is a hematopoietic stem cell. In embodiments, the hematopoietic stem cell is genetically modified to activate fetal hemoglobin. In embodiments the cell is a red blood cell. In embodiments, the red blood cell is genetically modified to activate fetal hemoglobin. For example, the cell may be a red blood cell genetically modified to activate fetal hemoglobin, wherein administration of the cell to a subject in need thereof treats or prevents thalassemia in the subject. In instances, the cell may be a red blood cell genetically modified to activate fetal hemoglobin, wherein administration of the cell to a subject in need thereof treats or prevents sickle cell anemia in the subject.

In an aspect is provided a cell made by a method provided herein including embodiments thereof. In an aspect is provided a population of cells made by a method provided herein including embodiments thereof.

### Methods of Making and Selecting Genetically Modified Cells

The nucleic acid construct provided herein including embodiments thereof is contemplated to be useful for selecting genetically modified cells from a population of cells. In embodiments, the population of cells includes a population of genetically modified and non-genetically modified cells. In embodiments, expression of tEGRF encoded by the nucleic acid construct provided herein allows for selection of the genetically modified cell by detection of the tEGFR protein. In embodiments, the genetically modified cell is modified by the gene editing agent (e.g. Cas9, TALEN, etc.) encoded by the nucleic acid construct provided herein including embodiments thereof. Thus, in an aspect is provided a method of selecting for genetically modified cells from a population of cells, the method including: (a) contacting the population of cells with a nucleic acid construct including a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent; (b) growing the cells under conditions such that: (i) the gene editing agent and the tEGFR are expressed in a subset of cells, and (ii) the gene editing agent edits one or more genes in the subset of the cells, thereby forming the genetically modified cells in the population of cells; and (c) selecting tEGFR-expressing cells, thereby selecting the genetically modified cells from the population of cells.

In embodiments, the method further includes isolating the genetically modified cells from the population of cells. The genetically modified cells can be isolated using any method known in the art. For example, the genetically modified cells can be isolated by FACS, antibody-based immunological methods (e.g. ELISA, etc.), magnetic-based cell sorting, etc.

In embodiments, the method further includes separating the genetically modified cells from the population of genetically modified and non-genetically modified cells. In embodiments, separating the cells includes selection of the genetically modified cells (e.g. by FACS, immunological antibody-based methods, etc.). In embodiments, the genetically modified cells can be selected and/or separated by detection of tEGFR expression. In embodiments, the population of cells do not express endogenous EGFR.

In embodiments, the method includes separating non-genetically modified cells from the population of cells including genetically modified and non-genetically modified cells. In embodiments, the population of cells includes about 5% to about 100% genetically modified cells after separating the non-genetically modified cells from the population of cells. In embodiments, the population of cells includes about 10% to about 100% genetically modified cells after separating the non-genetically modified cells from the population of cells. In embodiments, the population of cells includes about 15% to about 100% genetically modified cells after separating the non-genetically modified cells from the population of cells. In embodiments, the population of cells includes about 20% to about 100% genetically modified cells after separating the non-genetically modified cells from the population of cells. In embodiments, the population of cells includes about 25% to about 100% genetically modified cells after separating the non-genetically modified cells from the population of cells. In embodiments, the population of cells includes about 30% to about 100% genetically modified cells after separating the non-genetically modified cells from the population of cells. In embodiments, the population of cells includes about 35% to about 100% genetically modified cells after separating the non-genetically modified cells from the population of cells. In embodiments, the population of cells includes about 40% to about 100% genetically modified cells after separating the non-genetically modified cells from the population of cells. In embodiments, the population of cells includes about 45% to about 100% genetically modified cells after separating the non-genetically modified cells from the population of cells. In embodiments, the population of cells includes about 50% to about 100% genetically modified cells after separating the non-genetically modified cells from the population of cells. In embodiments, the population of cells includes about 55% to about 100% genetically modified cells after separating the non-genetically modified cells from the population of cells. In embodiments, the population of cells includes about 60% to about 100% genetically modified cells after separating the non-genetically modified cells from the population of cells. In embodiments, the population of cells includes about 65% to about 100% genetically modified cells after separating the non-genetically modified cells from the population of cells. In embodiments, the population of cells includes about 70% to about 100% genetically modified cells after separating the non-genetically modified cells from the population of cells. In embodiments, the population of cells includes about 75% to about 100% genetically modified cells after separating the non-genetically modified cells from the population of cells. In embodiments, the population of cells includes about 80% to about 100% genetically modified cells after separating the non-genetically modified cells from the population of cells. In embodiments, the population of cells includes about 85% to about 100% genetically modified cells after separating the non-genetically modified cells from the population of cells. In embodiments, the population of cells includes about 90% to about 100% genetically modified cells after separating the non-genetically modified cells from the population of cells. In embodiments, the population of cells includes about 95% to about 100% genetically modified cells after separating the non-genetically modified cells from the population of cells.

In embodiments, the population of cells includes about 5% to about 95% genetically modified cells after separating the non-genetically modified cells from the population of cells. In embodiments, the population of cells includes about 5% to about 90% genetically modified cells after separating the non-genetically modified cells from the population of cells. In embodiments, the population of cells includes about 5% to about 85% genetically modified cells after separating the non-genetically modified cells from the population of cells. In embodiments, the population of cells includes about 5% to about 80% genetically modified cells after separating the non-genetically modified cells from the population of cells. In embodiments, the population of cells includes about 5% to about 75% genetically modified cells after separating the non-genetically modified cells from the population of cells. In embodiments, the population of cells includes about 5% to about 70% genetically modified cells after separating the non-genetically modified cells from the population of cells. In embodiments, the population of cells includes about 5% to about 65% genetically modified cells after separating the non-genetically modified cells from the population of cells. In embodiments, the population of cells includes about 5% to about 60% genetically modified cells after separating the non-genetically modified cells from the population of cells. In embodiments, the population of cells includes about 5% to about 55% genetically modified cells after separating the non-genetically modified cells from the population of cells. In embodiments, the population of cells includes about 5% to about 50% genetically modified cells after separating the non-genetically modified cells from the population of cells. In embodiments, the population of cells includes about 5% to about 45% genetically modified cells after separating the non-genetically modified cells from the population of cells. In embodiments, the population of cells includes about 5% to about 40% genetically modified cells after separating the non-genetically modified cells from the population of cells. In embodiments, the population of cells includes about 5% to about 35% genetically modified cells after separating the non-genetically modified cells from the population of cells. In embodiments, the population of cells includes about 5% to about 20% genetically modified cells after separating the non-genetically modified cells from the population of cells. In embodiments, the population of cells includes about 5% to about 15% genetically modified cells after separating the non-genetically modified cells from the population of cells. In embodiments, the population of cells includes about 5% to about 10% genetically modified cells after separating the non-genetically modified cells from the population of cells. In embodiments, the population of cells includes about 5%, 10%, 15%, 20%, 25%, 30%, 35%, 40%, 45%, 50%, 55%, 60%, 65%, 70%, 75%, 80% 85%, 90%, 95%, or 100% genetically modified cells after separating the non-genetically modified cells from the population of cells.

For the method provided herein, in embodiments, the nucleic acid construct further includes a polynucleotide encoding a cleavage site. In embodiments, the cleavage site is a furin cleavage site. In embodiments, the furin cleavage site includes the amino acid sequence: RXXR, wherein X is a naturally occurring amino acid residue. In embodiments, the furin cleavage site includes the amino acid sequence: RRKR. For the method provided herein, in embodiments, the linker peptide is cleavable. In embodiments, the linker peptide includes a self-cleaving peptide. In embodiments, the self-cleaving peptide includes a T2A sequence, E2A sequence, P2A sequence, or F2A sequence. In embodiments, the nucleic acid construct further includes a promoter operably linked to the polynucleotide encoding the gene editing agent, the polynucleotide encoding the linker peptide, and the polynucleotide encoding the tEGFR. For the method provided herein, in embodiments, the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease. In embodiments, the gene editing agent includes an RNA-guided nuclease. In embodiments, the RNA-guided nuclease includes a Cas protein or variant thereof.

For the method provided herein including embodiments thereof, the polynucleotide encoding the tEGFR is 3′ of the polynucleotide encoding the linker peptide, and the polynucleotide encoding the cleavage site is 3′ of the polynucleotide encoding the gene editing agent. In embodiments, the polynucleotide encoding the gene editing agent is 3′ of the polynucleotide encoding the linker peptide, and the polynucleotide encoding the cleavage site is 3′ of the polynucleotide encoding the tEGFR. In embodiments, the nucleic acid construct includes a polynucleotide encoding a GSG linker. In embodiments, the GSG linker is 3′ of the polynucleotide encoding the cleavage site. For example, the GSG linker may be located between the self-cleaving site and the linker peptide. For the method provided herein, in embodiments, the polynucleotide encoding tEGFR includes a polynucleotide sequence of SEQ ID NO.:4. In embodiments, the nucleic acid construct includes the nucleic acid sequence of SEQ ID NO.:1. In embodiments, the nucleic acid construct is DNA. In embodiments, the nucleic acid construct is RNA.

The nucleic acid construct provided herein including embodiments thereof is contemplated to be useful for identifying genetically modified cells. For example, the genetically modified cells include a nucleic acid construct provided herein, and therefore express the tEGFR protein encoded by a polynucleotide within the nucleic acid construct. Thus, detection of tEGFR allows for identification of the genetically modified cells. In embodiments, the genetically modified cell does not express endogenous EGFR. Thus, in an aspect is provided a method of identifying genetically modified cells in a population of cells, wherein the genetically modified cells express a truncated epidermal growth factor receptor (tEGFR) and a gene editing agent, the method including detecting tEGFR-expressing cells, thereby identifying the genetically modified cells in the population of cells. In another aspect is provided a method for identifying genetically modified cells from a cell population, wherein at least a subset of the cells in the cell population have been genetically modified, and wherein the genetically modified cells express a truncated epidermal growth factor receptor (tEGFR). In embodiments, the method includes determining the amount of cells in the composition expressing the tEGFR.

For the method provided herein, including embodiments thereof, the genetically modified cells are made by: (a) contacting a population of cells with a nucleic acid construct provided herein including embodiments thereof, and (i) growing the cells under conditions such that: (ii) the gene editing agent and the tEGFR are expressed, and the gene editing agent edits one or more genes in the cells, thereby forming genetically modified cells. For the method provided herein, in embodiments, contacting the population of cells with the nucleic acid construct occurs under conditions such that the nucleic acid construct is delivered into the cell. Delivery of the nucleic acid construct into the cell can occur through any method known in the art. For example, in embodiments, contacting the cells with the nucleic acid construct includes electroporating the cells to deliver the nucleic acid construct into the cells. In embodiments, contacting the cells with the nucleic acid construct includes transfecting the cells to deliver the nucleic acid construct into the cells. In embodiments, contacting the cells with the nucleic acid construct includes chemically transfecting the cells to deliver the nucleic acid construct into the cells.

For the method provided herein, in embodiments, the cells are T cells. In embodiments, the T cells are genetically modified to express a chimeric antigen receptor. In embodiments, the T cells are genetically modified to inhibit endogenous CCR5 expression. In embodiments, the cells are red blood cells. In embodiments, the red blood cells are genetically modified to activate fetal hemoglobin. In embodiments, the cells are hematopoietic stem cells (HSCs). In embodiments, the HSCs are genetically modified to activate fetal hemoglobin.

### Methods of Release Testing Genetically Modified Cells

The nucleic acid construct provided herein including embodiments thereof is contemplated to be useful for release testing genetically-modified cells. As used herein, the term “release testing” refers to characterizing the identity and/or functionality (e.g. the activity of the gene editing agent (e.g. Cas9, TALEN, MegaTal, etc.) in the cell) and/or the amount of genetically modified cells in a population of cells, and determining that the cells are ready for release based on characterization of the cells. For example, in embodiments, the genetically modified cell is identified by detection of tEGFR expression. In, embodiments, if the amount of desired cells (e.g. genetically modified cells) is above a certain threshold in the population of cells, the population of cells is ready to be released. For example, in embodiments, if 5% of a population of cells are genetically modified cells (e.g. tEGFR expressing cells), the population of cells may be ready for release. Similarly, if the functionality of the genetically modified cell is confirmed (e.g. the genetic modification is detected in the cell, the genetic modification is quantified above a certain threshold in a population of cells, etc.) within a population of cells, the population of cells is ready to be released. As used herein, “release” refers to use of the cells (e.g. for in vitro testing in cells, for in vivo testing in an organism, for therapeutic purposes, for administration to a subject, etc.). Thus, in embodiments, releasing the cells is use of the cells for administration to a subject in need thereof. In embodiments, releasing the cells is use of the cells for in vitro testing. In embodiments, releasing the cells is use of the cells for in vivo testing in an organism. In embodiments, releasing the cells is use of the cells is use of the cells for therapeutic purposes.

Thus, in embodiments, release testing refers to characterizing the purity of a population of cells including genetically modified cells. In embodiments, the population of cells includes genetically modified cells and non-genetically modified cells. Characterization of the purify of the cells may include detecting the amount of genetically modified cells (e.g. by detection of tEGFR expression) in a population of cells including genetically modified and non-genetically modified cells. Detection of the genetically modified cells can be achieved by methods well known in the art (e.g. FACS, antibody-based immunological assays, etc.).

In embodiments, release testing refers to determining the functionality of the genetically modified cell. For example, determining functionality of the genetically modified cell may be detection of one or more modified genes within the cell. In embodiments, detection of one or more modified genes in a cell includes quantifying the frequency of the gene modification (e.g. insertion, deletion, point mutation, etc.). Detection of a modified gene can be achieved by methods known in the art, including but not limited to, tracking of indels by decomposition (TIDE), mismatch cleavage assay using T7 endonuclease, Sanger sequencing, next generation sequencing, etc. Methods for detecting and/or quantifying gene modifications in cells are described in references: Brinkman, E. K. et al. (2014) Easy quantitative assessment of genome editing by sequence trace decomposition. Nucleic Acids Res 42(22): e168. https://doi.org/10.1093/nar/gku936.; Brinkman, E. K. et al. (2018) Easy quantification of template-directed CRISPR/Cas9 editing. Nucleic Acids Res 46(10): e58. doi: 10.1093/nar/gky164.; Yang, Z. et al. (2015) Fast and sensitive detection of indels induced by precise gene targeting. Nucleic Acids Res 43(9): e59. doi: 10.1093/nar/gkvl26.; Hsiau, T. et al. (2018) Inference of CRISPR edits from Sanger trace data. bioRxiv. doi: 10.1101/251082.; Hodgens, C. et al. (2017) indCAPS: A tool for designing screening primers for CRISPR/Cas9 mutagenesis events. PLoS One 12(11): e0188406.https://doi.org/10.1371/joumal.pone.0188406.; Bell, C. et al. (2014) A high-throughput screening strategy for detecting CRISPR-Cas9 induced mutations using next-generation sequencing. BMC Genomics 15: 1002. https:/I/doi.org/10.1186/1471-2164-15-1002.; Sentmanat, M. F. et al. (2018) A survey of validation strategies for CRISPR-Cas9 editing. Sci Rep 8(1): 888. https://doi.org/10.1038/s41598-018-19441-8.; Pinello, L. et al. (2016) Analyzing CRISPR genome-editing experiments with CRISPResso. Nat Biotechnol 34(7): 695-7. https://doi.org/10.1038/nbt.3583.; Guell M et al. (2014) Genome editing assessment using CRISPR Genome Analyzer (CRISPR-GA). Bioinformatics 30(20): 2968-70. DOI: 10.1093/bioinformatics/btu427.; which are incorporated by reference herein in their entirety and for all purposes.

Thus, in an aspect is provided a method of release testing a population of cells comprising genetically modified cells, wherein the genetically modified cells express a truncated epidermal growth factor receptor (tEGFR) and a gene editing agent, the method including detecting an amount of tEGFR-expressing cells in the population of cells, wherein the population of cells is ready for release if at least about 2.5% of the cells in the cell population are tEGFR-expressing cells. In an aspect is provided a method of release testing a population of cells comprising genetically modified cells, wherein the genetically modified cells express a truncated epidermal growth factor receptor (tEGFR) and a gene editing agent, the method including detecting an amount of tEGFR-expressing cells in the population of cells, wherein the population of cells is ready for release if at least about 5% of the cells in the cell population are tEGFR-expressing cells.

In embodiments, the population of cells is ready for release if at least about 2.5% to about 100% of the cells in the cell population are tEGFR-expressing cells. In embodiments, the population of cells is ready for release if at least about 5% to about 100% of the cells in the cell population are tEGFR-expressing cells. In embodiments, the population of cells is ready for release if at least about 7.5% to about 100% of the cells in the cell population are tEGFR-expressing cells. In embodiments, the population of cells is ready for release if at least about 10% to about 100% of the cells in the cell population are tEGFR-expressing cells. In embodiments, the population of cells is ready for release if at least about 22.5% to about 100% of the cells in the cell population are tEGFR-expressing cells. In embodiments, the population of cells is ready for release if at least about 25% to about 100% of the cells in the cell population are tEGFR-expressing cells. In embodiments, the population of cells is ready for release if at least about 27.5% to about 100% of the cells in the cell population are tEGFR-expressing cells. In embodiments, the population of cells is ready for release if at least about 30% to about 100% of the cells in the cell population are tEGFR-expressing cells. In embodiments, the population of cells is ready for release if at least about 32.5% to about 100% of the cells in the cell population are tEGFR-expressing cells. In embodiments, the population of cells is ready for release if at least about 35% to about 100% of the cells in the cell population are tEGFR-expressing cells. In embodiments, the population of cells is ready for release if at least about 37.5% to about 100% of the cells in the cell population are tEGFR-expressing cells. In embodiments, the population of cells is ready for release if at least about 40% to about 100% of the cells in the cell population are tEGFR-expressing cells. In embodiments, the population of cells is ready for release if at least about 42.5% to about 100% of the cells in the cell population are tEGFR-expressing cells. In embodiments, the population of cells is ready for release if at least about 45% to about 100% of the cells in the cell population are tEGFR-expressing cells. In embodiments, the population of cells is ready for release if at least about 47.5% to about 100% of the cells in the cell population are tEGFR-expressing cells. In embodiments, the population of cells is ready for release if at least about 50% to about 100% of the cells in the cell population are tEGFR-expressing cells. In embodiments, the population of cells is ready for release if at least about 52.5% to about 100% of the cells in the cell population are tEGFR-expressing cells. In embodiments, the population of cells is ready for release if at least about 55% to about 100% of the cells in the cell population are tEGFR-expressing cells. In embodiments, the population of cells is ready for release if at least about 57.5% to about 100% of the cells in the cell population are tEGFR-expressing cells. In embodiments, the population of cells is ready for release if at least about 60% to about 100% of the cells in the cell population are tEGFR-expressing cells. In embodiments, the population of cells is ready for release if at least about 62.5% to about 100% of the cells in the cell population are tEGFR-expressing cells. In embodiments, the population of cells is ready for release if at least about 65% to about 100% of the cells in the cell population are tEGFR-expressing cells. In embodiments, the population of cells is ready for release if at least about 67.5% to about 100% of the cells in the cell population are tEGFR-expressing cells. In embodiments, the population of cells is ready for release if at least about 70% to about 100% of the cells in the cell population are tEGFR-expressing cells. In embodiments, the population of cells is ready for release if at least about 72.5% to about 100% of the cells in the cell population are tEGFR-expressing cells. In embodiments, the population of cells is ready for release if at least about 75% to about 100% of the cells in the cell population are tEGFR-expressing cells. In embodiments, the population of cells is ready for release if at least about 77.5% to about 100% of the cells in the cell population are tEGFR-expressing cells. In embodiments, the population of cells is ready for release if at least about 80% to about 100% of the cells in the cell population are tEGFR-expressing cells. In embodiments, the population of cells is ready for release if at least about 82.5% to about 100% of the cells in the cell population are tEGFR-expressing cells. In embodiments, the population of cells is ready for release if at least about 85% to about 100% of the cells in the cell population are tEGFR-expressing cells. In embodiments, the population of cells is ready for release if at least about 87.5% to about 100% of the cells in the cell population are tEGFR-expressing cells. In embodiments, the population of cells is ready for release if at least about 90% to about 100% of the cells in the cell population are tEGFR-expressing cells. In embodiments, the population of cells is ready for release if at least about 92.5% to about 100% of the cells in the cell population are tEGFR-expressing cells. In embodiments, the population of cells is ready for release if at least about 95% to about 100% of the cells in the cell population are tEGFR-expressing cells. In embodiments, the population of cells is ready for release if at least about 97.5% to about 100% of the cells in the cell population are tEGFR-expressing cells. Percentages may be any value or sub-range within the recited ranges, including endpoints.

In embodiments, the population of cells is ready for release if at least about 2.5% to about 97.5% of the cells in the cell population are tEGFR-expressing cells. In embodiments, the population of cells is ready for release if at least about 2.5% to about 95% of the cells in the cell population are tEGFR-expressing cells. In embodiments, the population of cells is ready for release if at least about 2.5% to about 92.5% of the cells in the cell population are tEGFR-expressing cells. In embodiments, the population of cells is ready for release if at least about 2.5% to about 90% of the cells in the cell population are tEGFR-expressing cells. In embodiments, the population of cells is ready for release if at least about 2.5% to about 87.5% of the cells in the cell population are tEGFR-expressing cells. In embodiments, the population of cells is ready for release if at least about 2.5% to about 85% of the cells in the cell population are tEGFR-expressing cells. In embodiments, the population of cells is ready for release if at least about 2.5% to about 82.5% of the cells in the cell population are tEGFR-expressing cells. In embodiments, the population of cells is ready for release if at least about 2.5% to about 80% of the cells in the cell population are tEGFR-expressing cells. In embodiments, the population of cells is ready for release if at least about 2.5% to about 77.5% of the cells in the cell population are tEGFR-expressing cells. In embodiments, the population of cells is ready for release if at least about 2.5% to about 75% of the cells in the cell population are tEGFR-expressing cells. In embodiments, the population of cells is ready for release if at least about 2.5% to about 72.5% of the cells in the cell population are tEGFR-expressing cells. In embodiments, the population of cells is ready for release if at least about 2.5% to about 70% of the cells in the cell population are tEGFR-expressing cells. In embodiments, the population of cells is ready for release if at least about 2.5% to about 67.5% of the cells in the cell population are tEGFR-expressing cells. In embodiments, the population of cells is ready for release if at least about 2.5% to about 65% of the cells in the cell population are tEGFR-expressing cells. In embodiments, the population of cells is ready for release if at least about 2.5% to about 62.5% of the cells in the cell population are tEGFR-expressing cells. In embodiments, the population of cells is ready for release if at least about 2.5% to about 60% of the cells in the cell population are tEGFR-expressing cells. In embodiments, the population of cells is ready for release if at least about 2.5% to about 57.5% of the cells in the cell population are tEGFR-expressing cells. In embodiments, the population of cells is ready for release if at least about 2.5% to about 55% of the cells in the cell population are tEGFR-expressing cells. In embodiments, the population of cells is ready for release if at least about 2.5% to about 52.5% of the cells in the cell population are tEGFR-expressing cells. In embodiments, the population of cells is ready for release if at least about 2.5% to about 50% of the cells in the cell population are tEGFR-expressing cells. In embodiments, the population of cells is ready for release if at least about 2.5% to about 47.5% of the cells in the cell population are tEGFR-expressing cells. In embodiments, the population of cells is ready for release if at least about 2.5% to about 45% of the cells in the cell population are tEGFR-expressing cells. In embodiments, the population of cells is ready for release if at least about 2.5% to about 42.5% of the cells in the cell population are tEGFR-expressing cells. In embodiments, the population of cells is ready for release if at least about 2.5% to about 40% of the cells in the cell population are tEGFR-expressing cells. In embodiments, the population of cells is ready for release if at least about 2.5% to about 37.5% of the cells in the cell population are tEGFR-expressing cells. In embodiments, the population of cells is ready for release if at least about 2.5% to about 35% of the cells in the cell population are tEGFR-expressing cells. In embodiments, the population of cells is ready for release if at least about 2.5% to about 32.5% of the cells in the cell population are tEGFR-expressing cells. In embodiments, the population of cells is ready for release if at least about 2.5% to about 30% of the cells in the cell population are tEGFR-expressing cells. In embodiments, the population of cells is ready for release if at least about 2.5% to about 27.5% of the cells in the cell population are tEGFR-expressing cells. In embodiments, the population of cells is ready for release if at least about 2.5% to about 25% of the cells in the cell population are tEGFR-expressing cells. In embodiments, the population of cells is ready for release if at least about 2.5% to about 22.5% of the cells in the cell population are tEGFR-expressing cells. In embodiments, the population of cells is ready for release if at least about 2.5% to about 20% of the cells in the cell population are tEGFR-expressing cells. In embodiments, the population of cells is ready for release if at least about 2.5% to about 17.5% of the cells in the cell population are tEGFR-expressing cells. In embodiments, the population of cells is ready for release if at least about 2.5% to about 15% of the cells in the cell population are tEGFR-expressing cells. In embodiments, the population of cells is ready for release if at least about 2.5% to about 12.5% of the cells in the cell population are tEGFR-expressing cells. In embodiments, the population of cells is ready for release if at least about 2.5% to about 10% of the cells in the cell population are tEGFR-expressing cells. In embodiments, the population of cells is ready for release if at least about 2.5% to about 7.5% of the cells in the cell population are tEGFR-expressing cells. In embodiments, the population of cells is ready for release if at least about 2.5% to about 5% of the cells in the cell population are tEGFR-expressing cells. In embodiments, the population of cells is ready for release if at least about 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5% 60%, 62.5%, 65%, 67.5%, 70%, 72.5%, 75%, 77.5%, 80%, 82.5%, 85%, 87.5%, 90%, 92.5%, 95%, 97.5% or 100% of the cells in the cell population are tEGFR-expressing cells. Percentages may be any value or sub-range within the recited ranges, including endpoints.

In embodiments, the population of cells is ready for release if at least about 6% of the cells in the cell population are tEGFR-expressing cells. In embodiments, the population of cells is ready for release if at least about 7% of the cells in the cell population are tEGFR-expressing cells. In embodiments, the population of cells is ready for release if at least about 8% of the cells in the cell population are tEGFR-expressing cells. In embodiments, the population of cells is ready for release if at least about 9% of the cells in the cell population are tEGFR-expressing cells. In embodiments, the population of cells is ready for release if at least about 10% of the cells in the cell population are tEGFR-expressing cells. In embodiments, the population of cells is ready for release if at least about 11% of the cells in the cell population are tEGFR-expressing cells. In embodiments, the population of cells is ready for release if at least about 12% of the cells in the cell population are tEGFR-expressing cells. In embodiments, the population of cells is ready for release if at least about 13% of the cells in the cell population are tEGFR-expressing cells. In embodiments, the population of cells is ready for release if at least about 14% of the cells in the cell population are tEGFR-expressing cells. In embodiments, the population of cells is ready for release if at least about 15% of the cells in the cell population are tEGFR-expressing cells. In embodiments, the population of cells is ready for release if at least about 20% of the cells in the cell population are tEGFR-expressing cells.

In an aspect is provided a method for release testing of a cell population, wherein at least a subset of the cells in the cell population have been genetically modified, and wherein the genetically modified cells express a truncated epidermal growth factor receptor (tEGFR). In embodiments, the method includes determining the amount of cells in the cell population expressing the tEGFR and determining that the cell population is ready for release if the number of cells expressing tEGFR is above a certain threshold. In embodiments, the population of cells is ready for release if between about 20% and about 100% of the cells in the cell population express tEGFR. In embodiments, the population of cells is ready for release if at least about 20% of the cells in the cell population express tEGFR. In embodiments, the population of cells is ready for release if at least about 25% of the cells in the cell population express tEGFR. In embodiments, the population of cells is ready for release if at least about 30% of the cells in the cell population express tEGFR. In embodiments, the population of cells is ready for release if at least about 35% of the cells in the cell population express tEGFR. In embodiments, the population of cells is ready for release if at least about 40% of the cells in the cell population express tEGFR. In embodiments, the population of cells is ready for release if at least about 45% of the cells in the cell population express tEGFR. In embodiments, the population of cells is ready for release if at least about 50% of the cells in the cell population express tEGFR. In embodiments, the population of cells is ready for release if at least about 55% of the cells in the cell population express tEGFR. In embodiments, the population of cells is ready for release if at least about 60% of the cells in the cell population express tEGFR. In embodiments, the population of cells is ready for release if at least about 65% of the cells in the cell population express tEGFR. In embodiments, the population of cells is ready for release if at least about 70% of the cells in the cell population express tEGFR. In embodiments, the population of cells is ready for release if at least about 75% of the cells in the cell population express tEGFR. In embodiments, the population of cells is ready for release if at least about 80% of the cells in the cell population express tEGFR. In embodiments, the population of cells is ready for release if at least about 85% of the cells in the cell population express tEGFR. In embodiments, the population of cells is ready for release if at least about 90% of the cells in the cell population express tEGFR. In embodiments, the population of cells is ready for release if at least about 95% of the cells in the cell population express tEGFR. In embodiments, the population of cells is ready for release if at least about 100% of the cells in the cell population express tEGFR. Values may be any value or subrange within the recited ranges, including endpoints.

For the method provided herein including embodiments thereof, the genetically modified cells are made by: (a) contacting a population of cells with the nucleic acid construct provided herein including embodiments thereof, and (b) growing the cells under conditions such that: i) the gene editing agent and the tEGFR are expressed, and (ii) the gene editing agent edits one or more genes in the cells, thereby forming genetically modified cells.

### Methods of Treatment

The compositions (e.g. nucleic acid constructs, genetically modified cells, etc.) provided herein, including embodiments thereof, are contemplated to be effective for treating diseases (e.g. cancer, HIV, thalassemia, sickle cell anemia, etc.). Thus, in an aspect is provided a method of treating a disease in a subject in need thereof, the method including administering to the subject a therapeutically effect amount of a cell provided herein including embodiments thereof. In embodiments, the cell is autologous to the subject. For example, the nucleic acid construct provided herein including embodiments thereof may be delivered into a patient-derived cell (e.g. autologous cell). Expression of the gene editing agent allows for genetic modification of the cell. In embodiments, tEGFR expression allows for identification and sorting of the genetically modified cell. In embodiments, the sorted genetically modified cell is administered to the patient (e.g. by re-infusion). In embodiments, the cells do not express endogenous EGFR. In embodiments, the cells are T cells and the subject has a disease treatable by administration of CAR-T cells. In embodiments, the subject has human immunodeficiency virus (HIV). In embodiments, the subject has cancer.

For example, the compositions provided herein are contemplated to be effective for treating a subject with sickle cell anemia (also known as sickle cell disease) or thalassemia. As used herein, “sickle cell anemia” is used in accordance with its common meaning in the art and refers to a genetic red blood cell disease, more specifically the hemoglobin that carries oxygen and distributes it throughout the body. In instances, this group of red blood cell diseases is characterized by a mutation on the globin beta that forms the adult hemoglobin that results causes the sickle phenotype (HbS). As used herein, “thalassemia” is used in accordance with its common meaning and refers to a group of genetic blood disorders characterized by decreased production of hemoglobin. The severity of thalassemia relates to how many of the globin genes (four alpha, two beta) are missing.

Thus, in embodiments, the method includes administering to the subject a therapeutically effect amount of a cell provided herein including embodiments thereof wherein subject has thalassemia. In embodiments, cell is a hematopoietic stem progenitor cell (HSPC). For example, HSPCs can be genetically modified to express gamma-globin. Thus, a red blood cell derived from a genetically modified HSPC may express fetal hemoglobin (e.g. instead of sickle hemoglobin). Thus, administration of an HSPC cell genetically modified to express hemoglobin may be effective for treating sickle cell anemia in a subject in need thereof. In embodiments, the method includes administering to the subject a therapeutically effect amount of a cell provided herein including embodiments thereof wherein subject has sickle cell anemia. A red blood cell derived from a genetically modified HSPC may have increased levels of functional hemoglobin. Thus, administration of a genetically modified HSPC may be effective for treating Thalassemia in a subject in need thereof.

Thus, in embodiments, the HSPC are genetically modified to activate fetal hemoglobin. In embodiments, the HSPC treat thalassemia in a subject in need thereof. In embodiments, the HSPC treat sickle cell anemia in a subject in need thereof. For example, the HSPC may differentiate to red blood cells. Thus, the red blood cells derived from the genetically-modified HSPC include gene modifications of the HSPCs. For example, the HSPC can be genetically modified to express gamma-globin (HBG) instead of Hb S. In embodiments, the cells are red blood cells. In embodiments, the red blood cells are genetically modified to activate fetal hemoglobin. In embodiments, the red blood cells treat thalassemia in a subject in need thereof. In embodiments, the red blood cells treat sickle cell anemia in a subject in need thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### P EMBODIMENTS

P Embodiment 1. A nucleic acid construct comprising a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent.

P Embodiment 2. The nucleic acid of P embodiment 1, further comprising a polynucleotide encoding a cleavage site.

P Embodiment 3. The nucleic acid of P embodiment 2, wherein the cleavage site is a furin cleavage site.

P Embodiment 4. The nucleic acid of P embodiment 3, wherein the furin cleavage site comprises the amino acid sequence: RXXR.

P Embodiment 5. The nucleic acid of P embodiment 4, wherein the furin cleavage site comprises the amino acid sequence: RRKR.

P Embodiment 6. The nucleic acid of any one of P embodiments 1 to 5, further comprising a promoter functionally linked to the polynucleotide encoding the gene editing agent, the polynucleotide encoding the linker peptide, and the polynucleotide encoding the tEGFR.

P Embodiment 7. The nucleic acid of P embodiment 1 or 2, wherein the tEGFR comprises an amino acid sequence of SEQ ID NO.:4.

P Embodiment 8. The nucleic acid of any one of P embodiments 1 to 7, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

P Embodiment 9. The nucleic acid of any one of P embodiments 1 to 7, wherein the gene editing agent comprises an RNA-guided nuclease.

P Embodiment 10. The method of P embodiment 9, wherein the RNA-guided nuclease comprises a Cas protein.

P Embodiment 11. The nucleic acid of any one of P embodiments 1 to 10, wherein the linker peptide is a cleavable linker.

P Embodiment 12. The nucleic acid of P embodiment 11, wherein the cleavable linker is a self-cleaving linker.

P Embodiment 13. The nucleic acid of any one of P embodiments 1 to 12, wherein the linker peptide comprises T2A, E2A, P2A, or F2A.

P Embodiment 14. The nucleic acid of any one of P embodiments 1 to 13, wherein the polynucleotide encoding the tEGFR is 3′ of the polynucleotide encoding the linker peptide, and the polynucleotide encoding the cleavable linker is 3′ of the polynucleotide encoding the gene editing agent.

P Embodiment 15. The nucleic acid of any one of P embodiments 1 to 13, wherein the polynucleotide encoding the gene editing agent is 3′ of the polynucleotide encoding the linker peptide, and the polynucleotide encoding the linker peptide is 3′ of the polynucleotide encoding the tEGFR.

P Embodiment 16. The nucleic acid of any one of P embodiments 1 to 15, further comprising a nucleotide encoding a GSG linker.

P Embodiment 17. The nucleic acid of P embodiment 16, wherein the GSG linker is 3′ of the polynucleotide encoding the furin cleavage site.

P Embodiment 18. The nucleic acid of any one of P embodiments 1 to 17, wherein the nucleic acid construct comprises the nucleic acid sequence of SEQ ID NO.:1.

P Embodiment 19. The nucleic acid of any one of P embodiments 1 to 18 which is DNA.

P Embodiment 20. The nucleic acid of any one of P embodiments 1 to 18 which is RNA.

P Embodiment 21. A vector comprising the nucleic acid of any one of P embodiments 1 to 20.

P Embodiment 22. The vector of P embodiment 21 which is a viral vector or a plasmid.

P Embodiment 23. A cell comprising the nucleic acid of any one of P embodiments 1 to 20 or the vector of P embodiment 21 or P embodiment 22.

P Embodiment 24. The cell of P embodiment 23, wherein the cell does not express endogenous EGFR.

P Embodiment 25. A method for genetically engineering cells in a cell population, the method comprising: (a) contacting the cell population with a genome editing agent and a polynucleotide encoding a truncated epidermal growth factor receptor (tEGFR), thereby forming genetically engineered cells within the cell population; (b) growing the cells under conditions that the tEGFR is expressed in the genetically engineered cells; and (c) sorting the genetically engineered cells from the cell population based on expression of tEGFR.

P Embodiment 26. The method of P embodiment 25, wherein step (a) comprises contacting the cell population with a nucleic acid comprising polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent.

P Embodiment 27. The method of P embodiment 25 or 26, wherein the nucleic acid further comprises a promoter functionally linked to the polynucleotide encoding the gene editing agent, the polynucleotide encoding the linker peptide, and the polynucleotide encoding the tEGFR.

P Embodiment 28. The method of any one of P embodiments 25 to 27, wherein the tEGFR comprises an amino acid sequence of SEQ ID NO.:4.

P Embodiment 29. The method of any one of P embodiments 25 to 28, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

P Embodiment 30. The method of any one of P embodiments 25 to 28, wherein the gene editing agent comprises an RNA-guided nuclease.

P Embodiment 31. The method of P embodiment 30, wherein the RNA-guided nuclease comprises a Cas protein.

P Embodiment 32. The method of any one of P embodiments 25 to 31, wherein the linker peptide is a cleavable linker.

P Embodiment 33. The method of P embodiment 32, wherein the cleavable linker is a self-cleaving linker.

P Embodiment 34. The method of any one of P embodiments 25 to 33, wherein the linker peptide comprises T2A, E2A, P2A, or F2A.

P Embodiment 35. The method of any one of P embodiments 25 to 34, wherein the nucleic acid construct comprises the nucleic acid sequence of SEQ ID NO.:1.

P Embodiment 36. The method of any one of P embodiments 25 to 35, wherein the nucleic acid construct is DNA.

P Embodiment 37. The method of any one of P embodiments 25 to 35, wherein the nucleic acid construct is RNA.

P Embodiment 38. The method of any one of P embodiments 25 to 36, wherein the polynucleotide encoding the tEGFR is 3′ of the polynucleotide encoding the linker peptide, and the polynucleotide encoding the linker peptide is 3′ of the polynucleotide encoding the gene editing agent.

P Embodiment 39. The method of any one of P embodiments 25 to 36, wherein the polynucleotide encoding the gene editing agent is 3′ of the polynucleotide encoding the linker peptide, and the polynucleotide encoding the linker peptide is 3′ of the polynucleotide encoding the tEGFR.

P Embodiment 40. The method of any one of P embodiments 25 to 39, further comprising a nucleotide encoding a GSG linker.

P Embodiment 41. The method of P embodiment 40, wherein the GSG linker is 3′ of the polynucleotide encoding the linker peptide.

P Embodiment 42. A method for release testing of a cell population, wherein at least a subset of the cells in the cell population have been genetically modified, and wherein the genetically modified cells express a truncated epidermal growth factor receptor (tEGFR), the method comprising determining the amount of cells in the cell population expressing the tEGFR and determining that the composition is ready for release.

P Embodiment 43. The method of P embodiment 42, wherein the cells were genetically modified by the method of any one of P embodiments 25 to 41.

P Embodiment 44. The method of P embodiment 42 or 43, wherein the composition is ready for release if at least about 20% of the cells in the composition express tEGFR.

P Embodiment 45. A method for identifying genetically engineered cells from a cell population, wherein at least a subset of the cells in the cell population have been genetically modified, and wherein the genetically modified cells express a truncated epidermal growth factor receptor (tEGFR), the method comprising determining the amount of cells in the composition expressing the tEGFR.

P Embodiment 46. The method of P embodiment 45, wherein the cells were genetically modified by the method of any one of P embodiments 25 to 41.

P Embodiment 47. The method of any one of P embodiments 25 to 46, wherein the cells are T cells.

P Embodiment 48. The method of P embodiment 47, wherein the T cells are genetically engineered to disrupt endogenous CCR5 expression.

P Embodiment 49. The method of P embodiment 47 or 48, wherein the T cells are genetically engineered to express a chimeric antigen receptor.

P Embodiment 50. The method of any one of P embodiments 25 to 46, wherein the cells are red blood cells.

P Embodiment 51. The method of P embodiment 50, wherein the red blood cells are genetically engineered to activate fetal hemoglobin.

P Embodiment 52. The method of any one of P embodiments 25 to 51, wherein the tEGFR comprises at least a portion of a linker peptide.

P Embodiment 53. The method of P embodiment 52, wherein the linker peptide comprises T2A, E2A, P2A, or F2A.

P Embodiment 54. A method for administering genetically engineered cells to a subject in need thereof, the method comprising obtaining a cell population made by the method of any one of P embodiments 25 to 53 to the subject.

P Embodiment 55. The method of P embodiment 54, wherein the cells are T cells and the subject has a disease treatable by administration of CAR-T cells.

P Embodiment 56. The method of P embodiment 54 to 55, wherein the subject has human immunodeficiency virus (HIV).

P Embodiment 57. The method of P embodiment 54, wherein the cells are hematopoietic stem cells genetically engineered to activate fetal hemoglobin.

P Embodiment 58. The method of P embodiment 57, wherein the cells treat thalassemia.

P Embodiment 59. The method of P embodiment 57, wherein the cells treat sickle cell anemia.

P Embodiment 60. The method of any one of P embodiments 25 to 29, wherein the cells do not express endogenous EGFR.

### EMBODIMENTS

Embodiment 1. A nucleic acid construct comprising a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent.

Embodiment 2. The nucleic acid construct of embodiment 1, further comprising a polynucleotide encoding a cleavage site.

Embodiment 3. The nucleic acid construct of embodiment 2, wherein the cleavage site is a furin cleavage site.

Embodiment 4. The nucleic acid construct of embodiment 3, wherein the furin cleavage site comprises the amino acid sequence: RXXR, wherein X is a naturally occurring amino acid residue.

Embodiment 5. The nucleic acid construct of embodiment 3 or 4, wherein the furin cleavage site comprises the amino acid sequence: RRKR.

Embodiment 6. The nucleic acid construct of any one of embodiments 1 to 5, wherein the linker peptide is cleavable.

Embodiment 7. The nucleic acid construct of any one of embodiments 1 to 6, wherein the linker peptide comprises a self-cleaving peptide.

Embodiment 8. The nucleic acid construct of embodiment 7, wherein the self-cleaving peptide comprises a T2A sequence, E2A sequence, P2A sequence, or F2A sequence.

Embodiment 9. The nucleic acid construct of any one of embodiments 1 to 8, further comprising a promoter operably linked to the polynucleotide encoding the gene editing agent, the polynucleotide encoding the linker peptide, and the polynucleotide encoding the tEGFR.

Embodiment 10. The nucleic acid construct of any one of embodiments 1 to 9, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

Embodiment 11. The nucleic acid construct of any one of embodiments 1 to 10, wherein the gene editing agent comprises an RNA-guided nuclease.

Embodiment 12. The nucleic acid construct of embodiment 11, wherein the RNA-guided nuclease comprises a Cas protein or variant thereof.

Embodiment 13. The nucleic acid construct of any one of embodiments 2 to 12, wherein the polynucleotide encoding the tEGFR is 3′ of the polynucleotide encoding the linker peptide, and the polynucleotide encoding the cleavage site is 3′ of the polynucleotide encoding the gene editing agent.

Embodiment 14. The nucleic acid construct of any one of embodiments 2 to 12, wherein the polynucleotide encoding the gene editing agent is 3′ of the polynucleotide encoding the linker peptide, and the polynucleotide encoding the cleavage site is 3′ of the polynucleotide encoding the tEGFR.

Embodiment 15. The nucleic acid construct of any one of embodiments 1 to 14, further comprising a nucleotide encoding a GSG linker.

Embodiment 16. The nucleic acid construct of embodiment 15, wherein the GSG linker is 3′ of the polynucleotide encoding the cleavage site.

Embodiment 17. The nucleic acid construct of any one of embodiments 1 to 16, wherein the polynucleotide encoding tEGFR comprises a polynucleotide sequence of SEQ ID NO.:4.

Embodiment 18. The nucleic acid construct of any one of embodiments 1 to 13, wherein the nucleic acid construct comprises the nucleic acid sequence of SEQ ID NO.:1.

Embodiment 19. The nucleic acid construct of any one of embodiments 1 to 18, wherein the construct is DNA.

Embodiment 20. The nucleic acid construct of any one of embodiments 1 to 18, wherein the construct is RNA.

Embodiment 21. An expression vector comprising the nucleic acid construct of any one of embodiments 1 to 20.

Embodiment 22. The expression vector of embodiment 21, wherein said vector is a viral vector or a plasmid.

Embodiment 23. A cell comprising the nucleic acid construct of any one of embodiments 1 to 20 or the expression vector of embodiment 21 or 22.

Embodiment 24. The cell of embodiment 23, wherein the cell does not express endogenous EGFR.

Embodiment 25. The cell of embodiment 23 or 24, wherein the cell is an immune cell.

Embodiment 26. The cell of embodiment 25, where in the immune cell is a T cell.

Embodiment 27. The cell of any embodiment 23 or 24, wherein the cell is a hematopoietic stem cell.

Embodiment 28. The cell of embodiment 27, wherein the hematopoietic stem cell is genetically modified to activate fetal hemoglobin.

Embodiment 29. A method of selecting for genetically modified cells from a population of cells, the method comprising: (a) contacting the population of cells with a nucleic acid construct comprising a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent; (b) growing the cells under conditions such that: (i) the gene editing agent and the tEGFR are expressed in a subset of cells, and (ii) the gene editing agent edits one or more genes in the subset of the cells, thereby forming the genetically modified cells in the population of cells; and (c) selecting tEGFR-expressing cells, thereby selecting the genetically modified cells from the population of cells.

Embodiment 30. The method of embodiment 29, wherein the nucleic acid construct further comprises a polynucleotide encoding a cleavage site.

Embodiment 31. The method of embodiment 30, wherein the cleavage site is a furin cleavage site.

Embodiment 32. The method of embodiment 32, wherein the furin cleavage site comprises the amino acid sequence: RXXR, wherein X is a naturally occurring amino acid residue.

Embodiment 33. The method of embodiment 31 or 32, wherein the furin cleavage site comprises the amino acid sequence: RRKR.

Embodiment 34. The method of any one of embodiments 29 to 33, wherein the linker peptide is cleavable.

Embodiment 35. The method of any one of embodiments 29 to 34, wherein the linker peptide comprises a self-cleaving peptide.

Embodiment 36. The method of embodiment 35, wherein the self-cleaving peptide comprises a T2A sequence, E2A sequence, P2A sequence, or F2A sequence.

Embodiment 37. The method of any one of embodiments 29 to 36, wherein the nucleic acid construct further comprises a promoter operably linked to the polynucleotide encoding the gene editing agent, the polynucleotide encoding the linker peptide, and the polynucleotide encoding the tEGFR.

Embodiment 38. The method of any one of embodiments 29 to 37, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

Embodiment 39. The method of any one of embodiments 29 to 38, wherein the gene editing agent comprises an RNA-guided nuclease.

Embodiment 40. The method of embodiment 39, wherein the RNA-guided nuclease comprises a Cas protein or variant thereof.

Embodiment 41. The method of any one of embodiments 30 to 40, wherein the polynucleotide encoding the tEGFR is 3′ of the polynucleotide encoding the linker peptide, and the polynucleotide encoding the cleavage site is 3′ of the polynucleotide encoding the gene editing agent.

Embodiment 42. The method of any one of embodiments 30 to 40, wherein the polynucleotide encoding the gene editing agent is 3′ of the polynucleotide encoding the linker peptide, and the polynucleotide encoding the cleavage site is 3′ of the polynucleotide encoding the tEGFR.

Embodiment 43. The method of any one of embodiments 29 to 42, further comprising a polynucleotide encoding a GSG linker.

Embodiment 44. The method of embodiment 43, wherein the GSG linker is 3′ of the polynucleotide encoding the cleavage site.

Embodiment 45. The method of any one of embodiments 29 to 44, wherein the polynucleotide encoding tEGFR comprises a polynucleotide sequence of SEQ ID NO.:4.

Embodiment 46. The method of any one of embodiments 29 to 41, wherein the nucleic acid construct comprises the nucleic acid sequence of SEQ ID NO.:1.

Embodiment 47. The method of any one of embodiments 29 to 46, wherein the nucleic acid construct is DNA.

Embodiment 48. The method of any one of embodiments 29 to 46, wherein the nucleic acid construct is RNA.

Embodiment 49. A method of release testing a population of cells comprising genetically modified cells, wherein the genetically modified cells express a truncated epidermal growth factor receptor (tEGFR) and a gene editing agent, the method comprising detecting an amount of tEGFR-expressing cells in the population of cells, wherein the population of cells is ready for release if at least about 2.5% cells in the cell population are tEGFR-expressing cells.

Embodiment 50. The method of embodiment 49, wherein the genetically modified cells are made by: (a) contacting a population of cells with the nucleic acid construct of any one of embodiments 1 to 20; and (b) growing the cells under conditions such that: (i) the gene editing agent and the tEGFR are expressed, and (ii) the gene editing agent edits one or more genes in the cells, thereby forming genetically modified cells.

Embodiment 51. A method of identifying genetically modified cells in a population of cells, wherein the genetically modified cells express a truncated epidermal growth factor receptor (tEGFR) and a gene editing agent, the method comprising detecting tEGFR-expressing cells, thereby identifying the genetically modified cells in the population of cells.

Embodiment 52. The method of embodiment 51, wherein the genetically modified cells are made by: (a) contacting a population of cells with the nucleic acid construct of any one of embodiments 1 to 20; and (b) growing the cells under conditions such that: (i) the gene editing agent and the tEGFR are expressed, and (ii) the gene editing agent edits one or more genes in the cells, thereby forming genetically modified cells.

Embodiment 53. The method of any one of embodiments 29 to 53, wherein the cells are T cells.

Embodiment 54. The method of embodiment 53, wherein the T cells are genetically modified to inhibit endogenous CCR5 expression.

Embodiment 55. The method of embodiment 53 or 54, wherein the T cells are genetically modified to express a chimeric antigen receptor.

Embodiment 56. The method of any one of embodiments 29 to 53, wherein the cells are red blood cells.

Embodiment 57. The method of embodiment 56, wherein the red blood cells are genetically modified to activate fetal hemoglobin.

Embodiment 58. A method of treating a disease in a subject in need thereof, the method comprising administering to the subject a therapeutically effect amount of the cells of any one of embodiments 23 to 28.

Embodiment 59. The method of embodiment 58, wherein the subject has a disease treatable by administration of CAR-T cells.

Embodiment 60. The method of embodiment 58 or 59, wherein the disease is human immunodeficiency virus (HIV), thalassemia or sickle cell anemia.

### EXAMPLES

One skilled in the art would understand that descriptions of making and using the particles described herein is for the sole purpose of illustration, and that the present disclosure is not limited by this illustration.

### Example 1. Concept and Initial Experiments

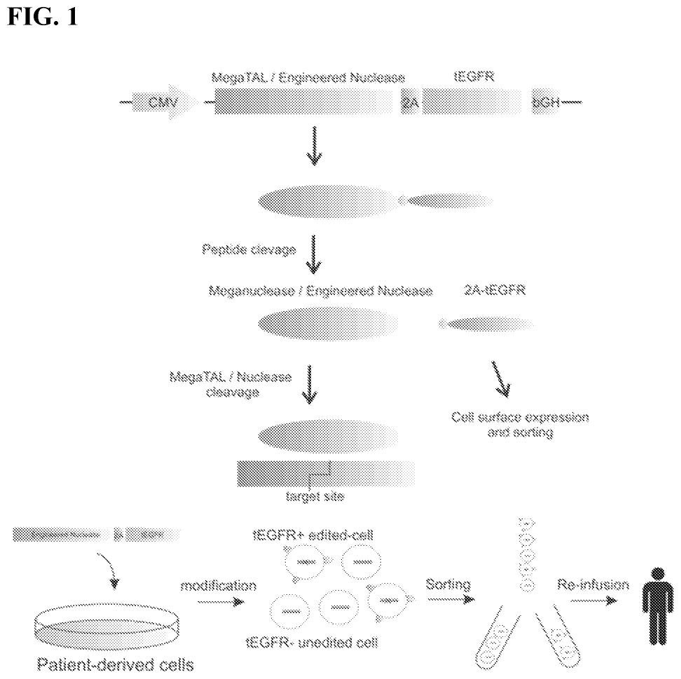

FIG. 1 shows a schematic of a construct comprising a MegaTAL, or an engineered nuclease, fused to a tEGFR domain by the means of a 2A cleavage peptide (2A). This system could be used to attach a variety of cell-surface markers to any engineered nuclease (e.g. CRISPR, Zinc-finger nuclease, TALEN, etc.) to sort edited cells. This enrichment would improve the quality as well as offer better control of the edited products for re-infusion into patients.

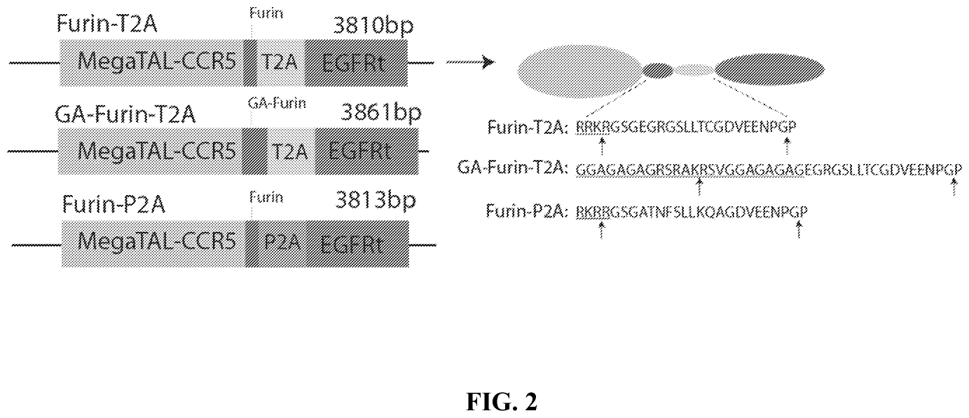

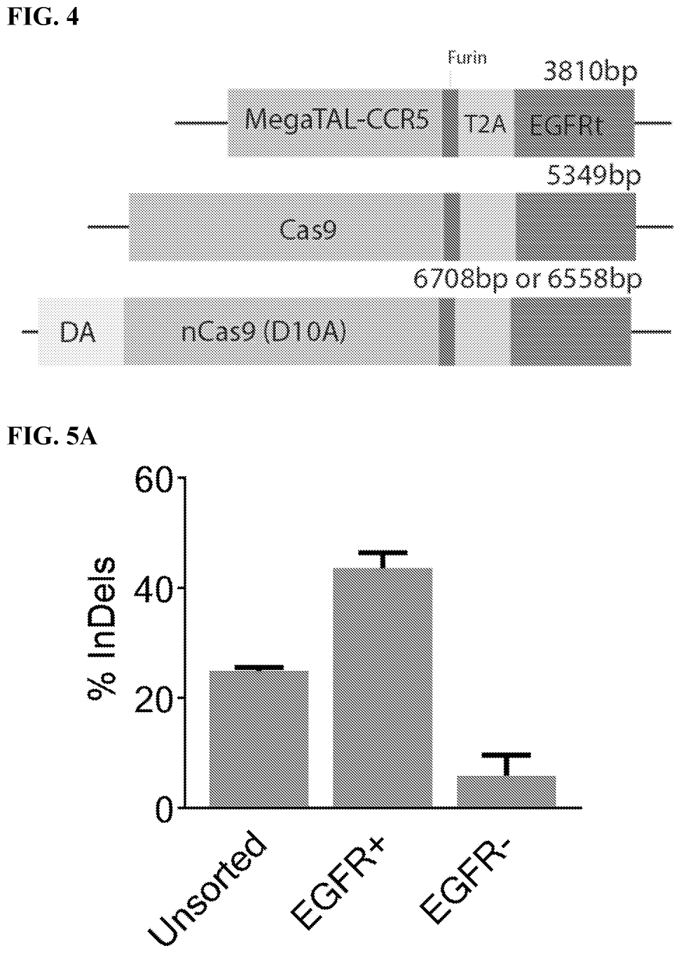

FIG. 2 and FIG. 4 show different constructs used in this study. The CCR5 gene-targeting MegaTAL (MT) (MegaTAL-CCR5) and tEGFR traffic to the nucleus or cell surface, respectively. To ensure separation, the two components were fused in-frame with the indicated furin cleavage domain and ribosomal skip peptide (T2A or P2A) separated by a GSG linker.

HEK293t cells were transfected with in vitro transcribed mRNA for expression of CCR5 targeting MegaTAL-tEGFR (CCR5MT-tEGFR) containing the furin T2A, furin P2A or GATA furin T2A linker. Cells were harvested after a 24 hr incubation and were subsequently surface stained for detection of tEGFR. Data was acquired on a Beckman Coulter Gallios flow cytometer and analyzed with FlowJo Software (formerly TreeStar, know BD Biosciences).

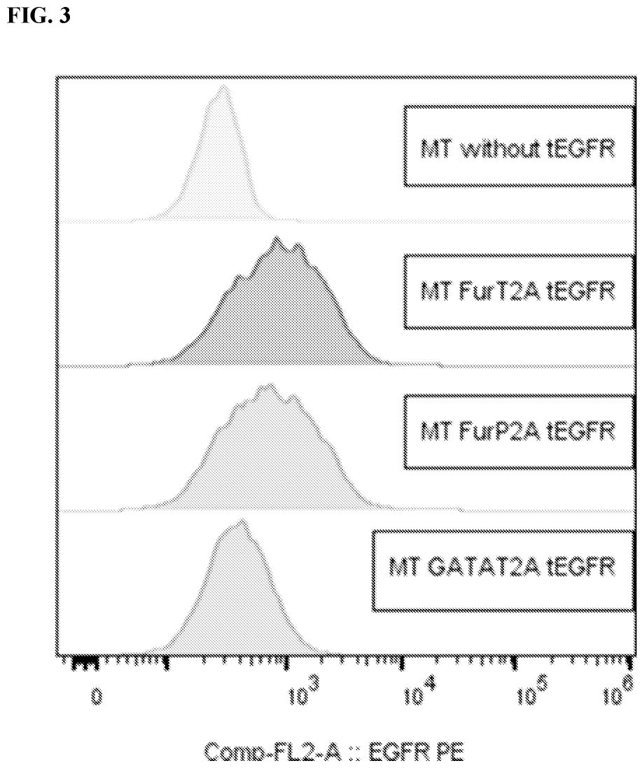

FIG. 3 shows cell-surface tEGFR expression using the constructs of FIG. 2, as evaluated in HEK293t cells. Results indicated that the constructs including furin T2A or P2A alone allowed for similar tEGFR expression on the cell surface. In contrast, use of the GATA furin T2A construct resulted in noticeably less tEGFR expression. This is visualized by the shift towards the left of the EGFR mean fluorescent peak on the histograms, indicating a lower level of tEGFR expression. These results suggested inefficient cleavage or translation of the tEGFR tag after translation of the MT, thus reducing the amount of tEGFR at the cell surface.

### Example 2. Effects of CCR5MT-EGFR Construct on Stable CCR5+ Cells

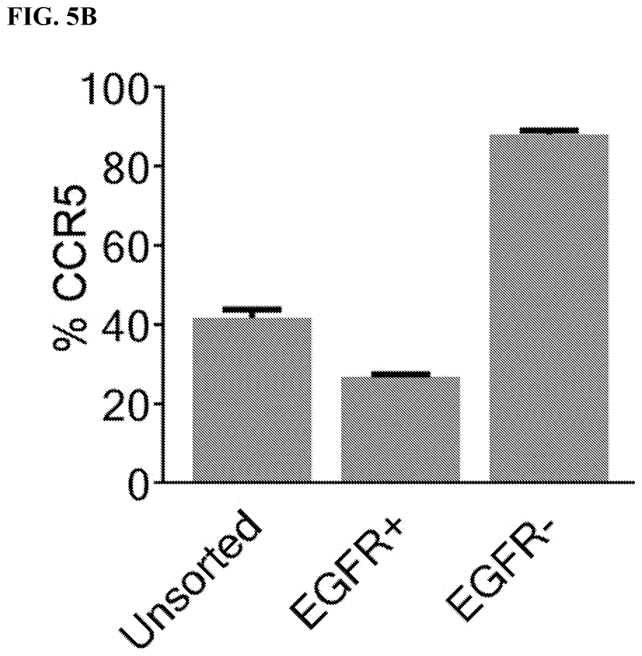

CEM.NKR CCR5+ cells were electroporated in triplicate with in vitro transcribed CCR5MT-tEGFR mRNA and incubated for 24 hours. The cells were then harvested and subsequently separated by tEGFR expression on a BD Fusion fluorescent cell sorter. A saved unsorted sample, tEGFR positive cells, and tEGFR negative cells were further cultured for another week and sampled for CCR5 gene disruption (FIG. 5A) and CCR5 surface expression (FIG. 5B). CCR5 gene disruption was determined by the frequency of insertions and deletions (InDels) detected after PCR amplification, sanger sequencing, and TIDE (tracking of indels by decomposition) analysis of the targeted CCR5 region. CCR5 surface expression was determined by flow cytometry on a Beckman Coulter Gallios and analyzed with FlowJo Software (formerly TreeStar, know BD Biosciences).

FIGS. 5A and 5B show the effects of the CCR5MT-tEGFR construct on a stable CCR5 expressing cell line (CEM.NKR CCR5+ cells). tEGFR expressing and positively sorted cells had approximately a two-fold increase in the frequency of InDels within the targeted region of CCR5 compared to unsorted cells. In accordance with the increase in InDel frequency, a decrease in CCR5 surface expression was also observed when cells are selected for EGFR expression.

### Example 3. Anti-HIV CAR T Cell Therapy

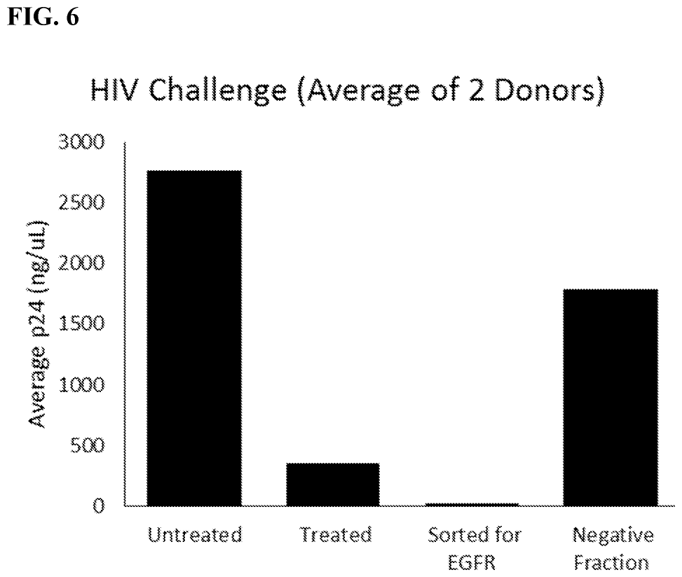

FIG. 6 shows results from an HIV challenge assay in which isolated human CD4 T cells were activated with T Cell Activation Media (CGD) for 48 hours at 37° C. prior to electroporation with CCR5MT-tEGFR mRNA. Cells were electroporated and then stained with CD3, CD4, and EGFR antibodies for separation. Cells were rested for four days and reactivated in TCA media for three days prior to R5 tropic HIV challenge (Bal). Serum was tested in triplicate at 7 days post challenge for p24 protein concentration as an indicator of HIV infection. Treated cells have a reduced susceptibility to R5 tropic HIV infection as indicated by the lower p24 concentration recovered from treated and tEGFR positive sorted samples after HIV challenge. The addition of the transient tEGFR surface tag for enrichment of cells successfully transfected with CCR5MT-EGFR mRNA, and improved the resistance of CD4 T cells to R5 tropic HIV infection least 500%. Table 1 below provides supporting data.

TABLE 1
Results of HIV Challenge Assay.

| | p24 ng/uL | | | |
|---|---|---|---|---|
| | | | Sorted for | Negative |
| HIV Challenge | Untreated | Treated | EGFR | fraction |
| Donor #1 | 181.00 | 455.67 | 1.67 | 674.67 |
| SEM | 8.19 | 38.67 | 0.33 | 12.44 |
| Donor #2 | 5342.33 | 244.67 | 41.67 | 2891.00 |
| SEM | 18.98 | 3.93 | 1.45 | 149.88 |

### Example 4. T7E1 InDel Detection Assay

The CCR5MT targeted region of CCR5 from bulk cultured T cells was PCR amplified followed by a melting and slow random reannealing of the unedited and edited DNA fragments. The mix was treated with T7 endonuclease 1 (T7E1) to cleave mismatched annealed fragments at the CCR5MT target site. The final product contains a mix of whole fragment (fragment A) and the fragment cleaved in two (fragments B and C). The mix is separated on an agarose gel and the concentration of each band determined by densitometry (Image Lab, Biorad). The frequency of cleaved DNA is the concentration of the two cleaved fragments (B+C) divided by the total concentration of DNA (A+B+C). The estimated InDels is an extended look establishing the potential error and is defined by 100×[1−(1−frequency of cleaved DNA)1/2].

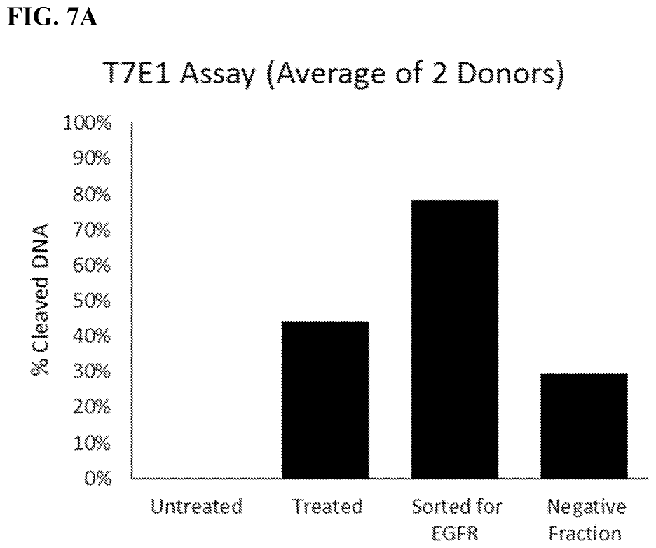

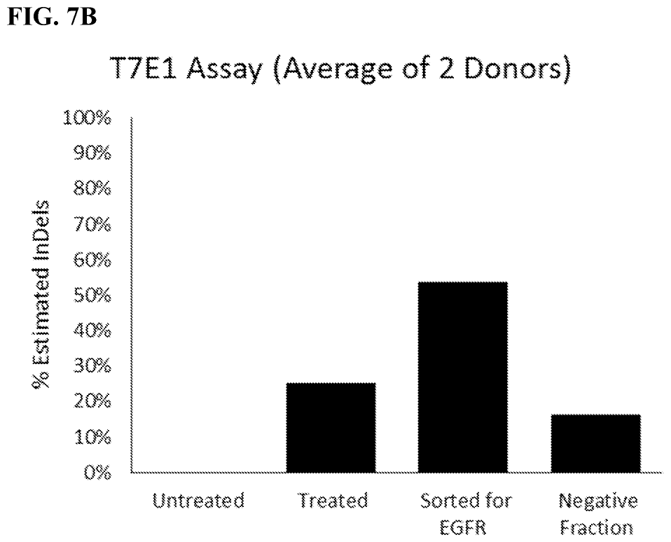

FIGS. 7A and 7B show graphs of a T7E1 assay for MegaTAL endonuclease activity. In this assay, InDels were undetectable from untreated cells which were also highly susceptible to R5 tropic HIV infection, while samples from cells exposed to CCR5MT-tEGFR had some degree of detectable InDels. The cells sorted for tEGFR had the highest frequency of InDels while the negative fraction had the lowest, suggesting the tEGFR tag allowed for rapid identification and isolation of cells with improved probability of nuclease activity. Table 2 below provides supporting data.

TABLE 2
Results of T7E1 InDel Detection Assay.
T7E1 InDel Detection Assay

| | Untreated | | Treated | | Sorted for EGFR | | Negative Fraction | |
|---|---|---|---|---|---|---|---|---|
| | Cleaved | Estimated | Cleaved | Estimated | Cleaved | Estimated | Cleaved | Estimated |
| | DNA | InDels | DNA | InDels | DNA | InDels | DNA | InDels |
| Donor #1 | 0.00% | 0.00% | 41.45% | 23.48% | 82.99% | 58.76% | 33.81% | 18.65% |
| Donor #2 | 0.00% | 0.00% | 46.70% | 27.00% | 73.54% | 48.56% | 24.92% | 13.35% |

### Example 5. Constructs with tEGFR Tag and Gene Editing Systems

K562 cells were electroporated with Cas9-tEGFR mRNA and sgRNA and incubated for 18 hours. Cells were surface stained for EGFR and data acquired on a BD LSRFortessa. Data was analyzed in FlowJo (Formerly TreeStar, now BD).

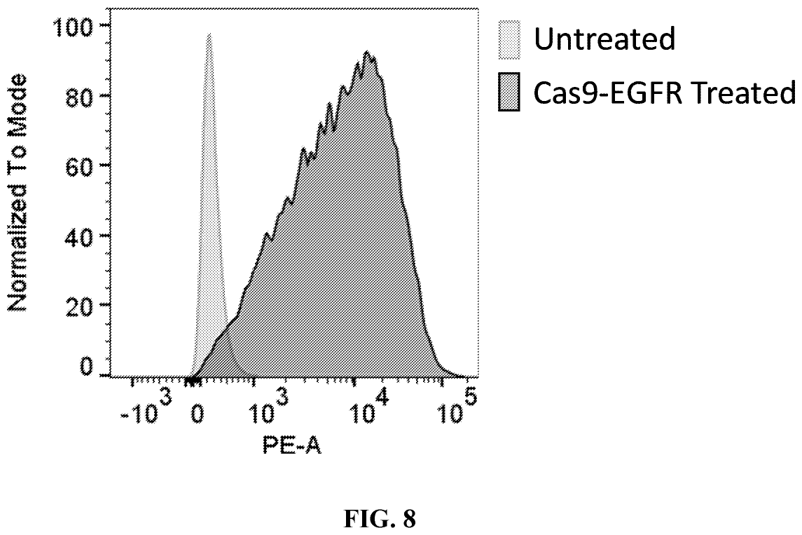

FIG. 8 shows FACS results of K562 cells electroporated with the Cas9-tEGFR construct as shown in FIG. 4, in which the tEGFR tag was fused to the Cas9 with a T2A ribosomal skip domain, in comparison to untreated K562 cells. The results of FIG. 8 show that the tEGFR tag could be used with other gene editing systems (here Cas9), when linked to the editing system by a T2A ribosomal skip domain.

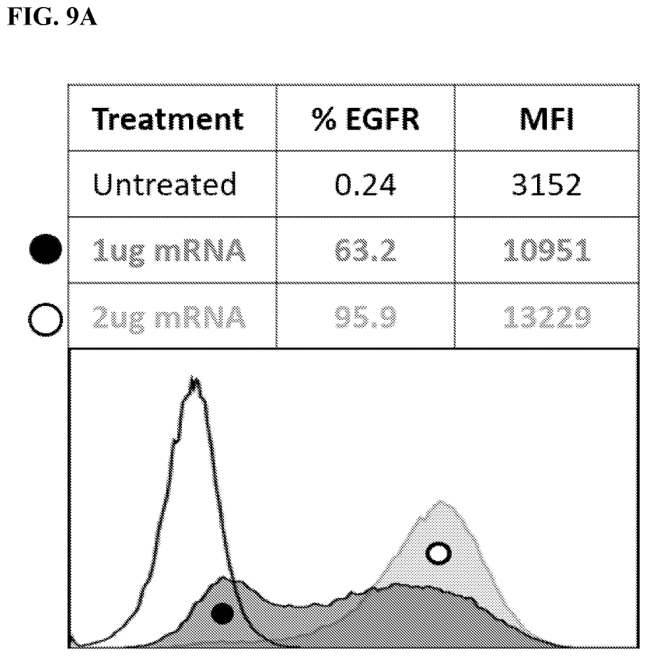

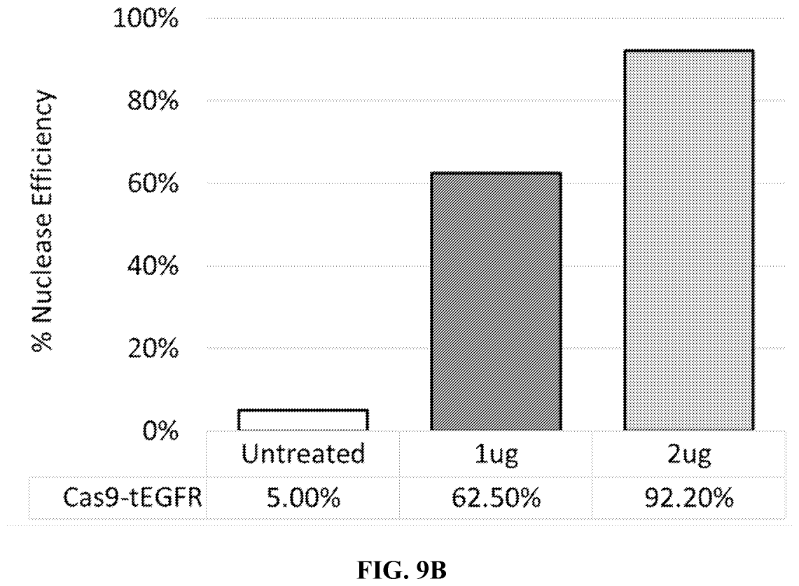

To further investigate the effects of Cas9-tEGFR, K562 cells were electroporated with two concentrations of the Cas9-tEGFR in combination with a targeting guide RNA. The cells were stained for cell surface tEGFR 18 hours later. Cells were cultured for an additional week prior to DNA extraction and TIDE assessment of the RNA guide targeted site. FIG. 9A shows tEGFR surface expression as determined by FACS (BD Fortessa). FIG. 9B shows InDel (insertion or deletion of bases) detection by TIDE assay. Significantly, and in accordance with other experiments described herein, tEGFR is detected in cells treated with the Cas9-tEGFR construct. Moreover, the cells treated with the Cas9-tEGFR construct had the highest frequency of InDels while the untreated cells had the lowest. Further, effects were shown to be dependent on the concentration of Cas9-tEGRF used for electroporation. Thus, these results show the gene editing construct allows for rapid identification of the cells with efficient gene editing.

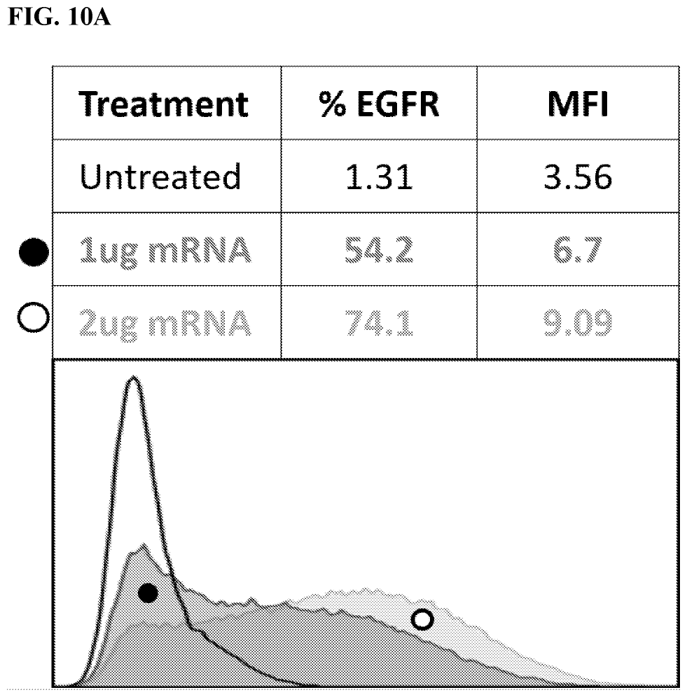

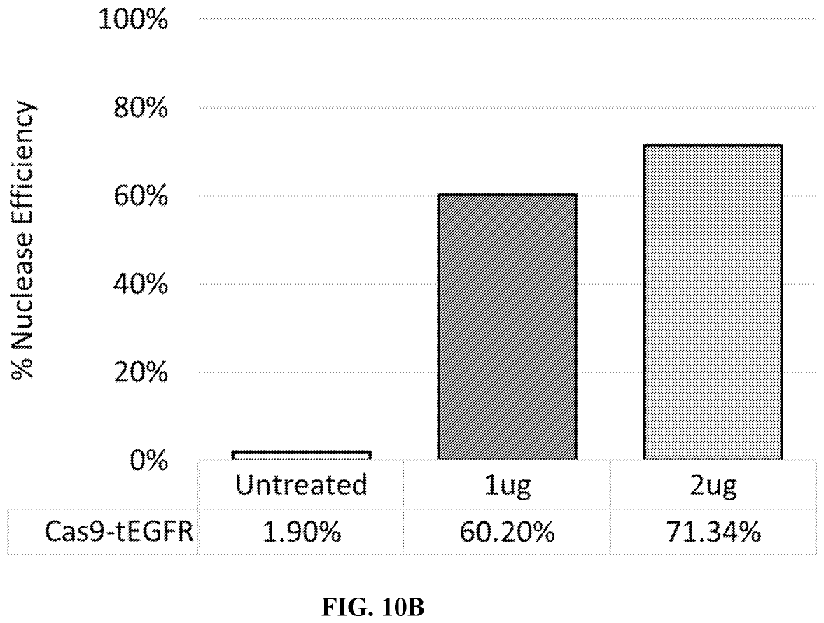

The effect of Cas9-tEGFR in activated T cells was then explored. Dynabead activated T cells were electroporated with two concentrations of the Cas9-tEGFR in combination with a targeting guide RNA and 18 hours later stained for cell surface tEGFR. Cells were cultured for an additional week prior to DNA extraction and TIDE assessment of the RNA guide targeted site. FIG. 10A shows tEGFR surface expression as determined by FACS (Miltenyi MACSQuant) and FIG. 10B shows InDel (insertion or deletion of bases) detection by TIDE assay. In accordance with other results described herein, tEGFR is detected in cells treated with the Cas9-tEGFR construct. Further, cell treatment with the construct results in efficient nuclease activity as revealed by TIDE analysis.

Thus, the results described show that tEGFR-tagged nuclease systems described herein can be used for both enrichment of gene-editing cells and assessment of the efficacy of nuclease delivery to target cells.

### REFERENCES

Hale, M., Mesojednik, T., Romano Ibarra, G., Sahni, J., Bernard, A., Sommer, K., Scharenberg, A., Rawlings, D. and Wagner, T., 2017. Engineering HIV-Resistant, Anti-HIV Chimeric Antigen Receptor T Cells. Molecular Therapy, 25(3), pp. 570-579.
Romano Ibarra, G., Paul, B., Sather, B., Younan, P., Sommer, K., Kowalski, J., Hale, M., Stoddard, B., Jarjour, J., Astrakhan, A., Kiem, H. and Rawlings, D., 2016. Efficient Modification of the CCR5 Locus in Primary Human T Cells With megaTAL Nuclease Establishes HIV-1 Resistance. Molecular Therapy—Nucleic Acids, 5, p.e352.
Sather, B., Romano Ibarra, G., Sommer, K., Curinga, G., Hale, M., Khan, I., Singh, S., Song, Y., Gwiazda, K., Sahni, J., Jarjour, J., Astrakhan, A., Wagner, T., Scharenberg, A. and Rawlings, D., 2015. Efficient modification of CCR5 in primary human hematopoietic cells using a megaTAL nuclease and AAV donor template. Science Translational Medicine, 7(307), pp. 307ra156-307ra156.
Wang, X., Chang, W., Wong, C., Colcher, D., Sherman, M., Ostberg, J., Forman, S., Riddell, S. and Jensen, M., 2011. A transgene-encoded cell surface polypeptide for selection, in vivo tracking, and ablation of engineered cells. Blood, 118(5), pp. 1255-1263.
Wang, X., Chang, W., Wong, C., Colcher, D., Sherman, M., Ostberg, J., Riddell, S., Forman, S. and Jensen, M., 2011. 38. A Transgene Encoded Cell Surface EGFR Polypeptide for Selection, In Vivo Tracking and Ablation of Engineered Cells. Molecular Therapy, 19, p.S15.
U.S. Pat. No. 8,802,374 B2.

What is claimed is:

1. A nucleic acid construct comprising a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent.

2. The nucleic acid construct of claim 1, further comprising a polynucleotide encoding a cleavage site.

3. The nucleic acid construct of claim 2, wherein the cleavage site is a furin cleavage site.

4. The nucleic acid construct of claim 3, wherein the furin cleavage site comprises the amino acid sequence: RXXR, wherein X is a naturally occurring amino acid residue.

5. The nucleic acid construct of claim 3, wherein the furin cleavage site comprises the amino acid sequence: RRKR.

6. The nucleic acid construct of claim 1, wherein the linker peptide is cleavable.

7. The nucleic acid construct of claim 1, wherein the linker peptide is comprises a self-cleaving peptide.

8. The nucleic acid construct of claim 7, wherein the self-cleaving peptide comprises a T2A sequence, E2A sequence, P2A sequence, or F2A sequence.

9. The nucleic acid construct of any one of claim 1, further comprising a promoter operably linked to the polynucleotide encoding the gene editing agent, the polynucleotide encoding the linker peptide, and the polynucleotide encoding the tEGFR.

10. The nucleic acid construct of any one of claims 1 to 9, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

11. The nucleic acid construct of claim 1, wherein the gene editing agent comprises an RNA-guided nuclease.

12. The nucleic acid construct of claim 11, wherein the RNA-guided nuclease comprises a Cas protein or variant thereof.

13. The nucleic acid construct of claim 2, wherein the polynucleotide encoding the tEGFR is 3′ of the polynucleotide encoding the linker peptide, and the polynucleotide encoding the cleavage site is 3′ of the polynucleotide encoding the gene editing agent.

14. The nucleic acid construct of claim 2, wherein the polynucleotide encoding the gene editing agent is 3′ of the polynucleotide encoding the linker peptide, and the polynucleotide encoding the cleavage site is 3′ of the polynucleotide encoding the tEGFR.

15. The nucleic acid construct of claim 1, further comprising a nucleotide encoding a GSG linker.

16. The nucleic acid construct of claim 15, wherein the GSG linker is 3′ of the polynucleotide encoding the cleavage site.

17. The nucleic acid construct of claim 1, wherein the polynucleotide encoding tEGFR comprises a polynucleotide sequence of SEQ ID NO.:4.

18. The nucleic acid construct of claim 1, wherein the nucleic acid construct comprises the nucleic acid sequence of SEQ ID NO.:1.

19. The nucleic acid construct of claim 1, wherein the construct is DNA.

20. The nucleic acid construct of claim 1, wherein the construct is RNA.

21. An expression vector comprising the nucleic acid construct of claim 1.

22. The expression vector of claim 21, wherein said vector is a viral vector or a plasmid.

23. A cell comprising the nucleic acid construct of claim 1 or the expression vector of claim 21.

24. The cell of claim 23, wherein the cell does not express endogenous EGFR.

25. The cell of claim 23, wherein the cell is an immune cell.

26. The cell of claim 25, where in the immune cell is a T cell.

27. The cell of any claim 23, wherein the cell is a hematopoietic stem cell.

28. The cell of claim 27, wherein the hematopoietic stem cell is genetically modified to activate fetal hemoglobin.

29. A method of selecting for genetically modified cells from a population of cells, the method comprising:
(a) contacting the population of cells with a nucleic acid construct comprising a polynucleotide encoding a gene editing agent, a polynucleotide encoding a linker peptide, and a polynucleotide encoding a truncated epidermal growth factor receptor (tEGFR), wherein the polynucleotide encoding the linker peptide is located between the polynucleotide encoding the tEGFR and the polynucleotide encoding the gene editing agent;
(b) growing the cells under conditions such that:
i) the gene editing agent and the tEGFR are expressed in a subset of cells, and
ii) the gene editing agent edits one or more genes in the subset of the cells, thereby forming the genetically modified cells in the population of cells; and
(c) selecting tEGFR-expressing cells, thereby selecting the genetically modified cells from the population of cells.

30. The method of claim 29, wherein the nucleic acid construct further comprises a polynucleotide encoding a cleavage site.

31. The method of claim 30, wherein the cleavage site is a furin cleavage site.

32. The method of claim 32, wherein the furin cleavage site comprises the amino acid sequence: RXXR, wherein X is a naturally occurring amino acid residue.

33. The method of claim 31, wherein the furin cleavage site comprises the amino acid sequence: RRKR.

34. The method of claim 29, wherein the linker peptide is cleavable.

35. The method of claim 29, wherein the linker peptide is comprises a self-cleaving peptide.

36. The method of claim 35, wherein the self-cleaving peptide comprises a T2A sequence, E2A sequence, P2A sequence, or F2A sequence.

37. The method of claim 29, wherein the nucleic acid construct further comprises a promoter operably linked to the polynucleotide encoding the gene editing agent, the polynucleotide encoding the linker peptide, and the polynucleotide encoding the tEGFR.

38. The method of claim 29, wherein the gene editing agent is a meganuclease, a clustered regularly interspaced short palindromic repeats (CRISPR) protein, a transcription activator-like effector nuclease (TALEN), a zinc finger nuclease, MegaTAL, or an Argonaute endonuclease.

39. The method of claim 29, wherein the gene editing agent comprises an RNA-guided nuclease.

40. The method of claim 39, wherein the RNA-guided nuclease comprises a Cas protein or variant thereof.

41. The method of claim 30, wherein the polynucleotide encoding the tEGFR is 3′ of the polynucleotide encoding the linker peptide, and the polynucleotide encoding the cleavage site is 3′ of the polynucleotide encoding the gene editing agent.

42. The method of claim 30, wherein the polynucleotide encoding the gene editing agent is 3′ of the polynucleotide encoding the linker peptide, and the polynucleotide encoding the cleavage site is 3′ of the polynucleotide encoding the tEGFR.

43. The method of claim 29, further comprising a polynucleotide encoding a GSG linker.

44. The method of claim 43, wherein the GSG linker is 3′ of the polynucleotide encoding the cleavage site.

45. The method of claim 29, wherein the polynucleotide encoding tEGFR comprises a polynucleotide sequence of SEQ ID NO.:4.

46. The method of claim 29, wherein the nucleic acid construct comprises the nucleic acid sequence of SEQ ID NO.:1.

47. The method of claim 29, wherein the nucleic acid construct is DNA.

48. The method of claim 29, wherein the nucleic acid construct is RNA.

49. A method of release testing a population of cells comprising genetically modified cells, wherein the genetically modified cells express a truncated epidermal growth factor receptor (tEGFR) and a gene editing agent, the method comprising detecting an amount of tEGFR-expressing cells in the population of cells, wherein the population of cells is ready for release if at least about 2.5% cells in the cell population are tEGFR-expressing cells.

50. The method of claim 49, wherein the genetically modified cells are made by:
(a) contacting a population of cells with the nucleic acid construct of claim 1; and
(b) growing the cells under conditions such that:
i) the gene editing agent and the tEGFR are expressed, and
ii) the gene editing agent edits one or more genes in the cells, thereby forming genetically modified cells.

51. A method of identifying genetically modified cells in a population of cells, wherein the genetically modified cells express a truncated epidermal growth factor receptor (tEGFR) and a gene editing agent, the method comprising detecting tEGFR-expressing cells, thereby identifying the genetically modified cells in the population of cells.

52. The method of claim 51, wherein the genetically modified cells are made by:
(a) contacting a population of cells with the nucleic acid construct of claim 1; and
(b) growing the cells under conditions such that:
i) the gene editing agent and the tEGFR are expressed, and
ii) the gene editing agent edits one or more genes in the cells, thereby forming genetically modified cells.

53. The method of claim 29, wherein the cells are T cells.

54. The method of claim 53, wherein the T cells are genetically modified to inhibit endogenous CCR5 expression.

55. The method of claim 54, wherein the T cells are genetically modified to express a chimeric antigen receptor.

56. The method of claim 29, wherein the cells are red blood cells.

57. The method of claim 56, wherein the red blood cells are genetically modified to activate fetal hemoglobin.

58. A method of treating a disease in a subject in need thereof, the method comprising administering to the subject a therapeutically effect amount of the cells of claim 23.

59. The method of claim 58, wherein the subject has a disease treatable by administration of CAR-T cells.

60. The method of claim 58, wherein the disease is human immunodeficiency virus (HIV), thalassemia or sickle cell anemia.
